SEQUENCE LISTING (9 sequences)

SEQ ID NO: 1 - protein, 9 aa, Artificial
  Synthetic peptide
SPSYVYHQF

SEQ ID NO: 2 - DNA, 27 bp, Artificial
  Synthetic primer
tattccggaggacccggacctatgaaa

SEQ ID NO: 3 - DNA, 33 bp, Artificial
  Synthetic reverse primer
taatgcggccgcttagtcgttggtccaaccttc

SEQ ID NO: 4 - protein, 9 aa, Friend murine leukemia virus
CCLCLTVFL

SEQ ID NO: 5 - protein, 21 aa, Artificial
  Synthetic peptide
FNNFTVSFWLRVPKVSASHLE

SEQ ID NO: 6 - protein, 9 aa, Artificial
  Synthetic peptide
WMHHNMDLI

SEQ ID NO: 7 - protein, 8 aa, Artificial
  Synthetic peptide
SNWYFNHL

SEQ ID NO: 8 - DNA, 31 bp, Artificial
  Synthetic primer
ttttaagcttgccgccaccatgggttggagc

SEQ ID NO: 9 - DNA, 66 bp, Artificial
  Synthetic reverse primer
ttttgcggccgcttacagaaaaacagtcaaacagagacaacagttaccccagaagtcacgcaggaa

(19) United States
(12) Patent Application Publication
(10) Pub. No.: US 2007/0253969 A1
(43) Pub. Date: Nov. 1, 2007

(54) Materials and methods relating to DNA vaccination

(76) Inventors: Freda Stevenson, Southampton (GB); Jason Rice, Southampton (GB); Sarah Buchan, Southampton (GB)

Correspondence Address: MORGAN LEWIS & BOCKIUS LLP, 1111 PENNSYLVANIA AVENUE NW, WASHINGTON, DC, 20004, US

(21) Appl. No.: 11/598,689

(22) Filed: Nov. 14, 2006

Related U.S. Application Data
(60) Provisional application No. 60/735,887, filed on Nov. 14, 2005.

(51) Int. Cl. A61K 39/00

(52) U.S. Cl. 424/184.1

(57) ABSTRACT

The present invention provides improved methods relating to DNA vaccination, particularly in relation to boosting the immune response. The inventors provide methods of immunizing an individual against an antigen, e.g. a tumor antigen using electroporation as a method of administration.

### RELATED APPLICATIONS

This application claims the benefit of U.S. Provisional Application No. 60/735,887 (filed Nov. 14, 2005) which is hereby incorporated by reference in its entirety.

### FIELD OF THE INVENTION

The present invention concerns materials and methods relating to DNA vaccination. Particularly, but not exclusively, the invention provides improved methods for immunizing a person against an antigen, preferably a tumor associated antigen using electroporation as a method of administration.

### BACKGROUND OF THE INVENTION

Deoxyribonucleic acid vaccination is emerging as an effective and safe strategy for inducing protective immunity in preclinical models of infectious disease (1-4), cancer (5-8), and autoimmunity (9-11). U.S. patent application Ser. No. 09/896,535 (WO94/08008), U.S. patent application Ser. No. 10/257,657 (WO01/79510) and U.S. patent application U.S. Ser. No. 10/416,290 (WO 02/40513), all of which are incorporated by reference, describe techniques for DNA vaccination. It is evident that DNA vaccines have the ability to stimulate a broad spectrum of immunological activities (12). In mice, they are capable of inducing potent cell-mediated and humoral immunity, although they are typically weaker at promoting antibody (Ab) responses than protein-based vaccines (3, 13). However, transferring this technology into large animals or human subjects has generally produced only modest results (14, 15).

Nevertheless, DNA vaccines are effective at priming immune responses in humans and large animals, a quality that can be exploited using the heterologous prime/boost approach, whereby the initial immune response to naked DNA vaccination is boosted by delivery of the same antigen (Ag) in a different vaccine vehicle (e.g., via viral or bacterial vectors, or as protein) (16-18). However, despite a large body of evidence in animal models and in the clinic (19) demonstrating the efficacy of heterologous prime/boost procedures, this approach has its limitations (20). Additional vectors or proteins raise regulatory and manufacturing issues. Importantly, preexisting or induced blocking immunity against the viral or bacterial vector is a major concern, especially for patients with cancer, in which it is likely that the vaccination program will be prolonged.

The inventors have previously developed DNA fusion vaccines, encoding tumor Ags linked to pathogen-derived sequences aimed to provide CD4⁺ T cell help critical for the induction and maintenance of antitumor immunity (20, 21, WO94/08008; WO01/79510 and WO02/40513—all incorporated by reference). By using the fragment C sequence of tetanus toxin, they can activate robust tumor-specific Ab, CD4⁺, and CD8⁺ T cell responses and protect mice from tumor (13, 22-24). However, because vaccine dose and volume, known to be critical for responses in mice (25-27), are difficult to scale up for human subjects, the inventors have appreciated that other delivery strategies are required.

Accordingly, there is a need for an improved method of delivering nucleic acid vaccines to human and larger animals. Important factors are the level of Ag expression and activation of innate immunity (12, 28). Numerous techniques are being developed to increase efficiency (28), with electroporation being particularly attractive, as it has been shown to increase DNA uptake and protein expression in various tissues in vivo (29-31). Improvement in vaccine potency has been observed in small and large animal models of infectious disease (26, 27, 32, 33).

### SUMMARY OF THE INVENTION

The inventors have applied the technique of electroporation to two models, the CT26 carcinoma and the BCL₁ lymphoma, susceptible to attack via either CD8⁺ T cells or Ab, respectively (23, 34, 35). They demonstrate an increase in priming by electroporation in both. Importantly, they show that a prime/boost approach with naked DNA, followed by DNA plus electroporation, amplifies both effector functions. Thus, for the first time, the inventors have shown conclusively that the induction of effective tumor-specific immunity is now feasible in cancer patients using only a single naked DNA vaccine format.

Specifically, the present inventors have tested electroporation as a method to increase the transfection efficiency and immune responses by tumor vaccines in vivo in mice. Using a DNA vaccine expressing the CTL epitope AH1 from colon carcinoma CT26, the inventors were able to confirm that effective priming and tumor protection in mice is highly dependent on vaccine dose and volume. However, the inventors have surprisingly determined that suboptimal vaccination was rendered effective by electroporation which primed higher levels of AH1-specific CD8+ T cell able to protect mice from tumor growth. Electroporation during priming with the inventor's optimal vaccination protocol did not improve CD8+ T cell responses. In contrast, electroporation during boosting strikingly improved vaccine performance. The prime/boost strategy was also effective if electroporation was used at both priming and boosting. For Ab production, DNA vaccination is generally less effective than protein. However, prime/boost with naked DNA followed by electroporation dramatically increased Ab levels. Thus, the priming qualities of DNA fusion vaccines, integrated with the improved Ag expression offered by electroporation, can be combined in a novel homologous prime/boost approach, to generate superior antitumor immune responses.

The present inventors have also modelled performance against a leukemia-associated antigen in a tolerized setting, by constructing a fusion vaccine encoding an immunodominant CTL epitope derived from Friend Murine Leukemia Virus gag protein (FMuLV_{gag}) and using it to vaccinate gag transgenic mice. Using a boost approach with DNA plus electroporation, the inventors were able to show that vaccination can activate surviving epitope-specific CTL remaining in a tolerized repertoire. Moreover, no evidence of autoimmune injury was seen.

Accordingly, at its most general, the present invention provides materials and methods for improved induction of an immune response in a mammal, preferably a human, using electroporation as the mode of administration.

In a first aspect, there is provided a method of boosting an immune response in an individual to an antigen, said individual having been previously primed against or exposed to said antigen, said method comprising administering to said individual a nucleic acid construct encoding said antigen by electroporation.

The individual may have been previously exposed to said antigen either naturally or by administration of the antigen either as a polypeptide or as a nucleic acid construct encoding said antigen. The previous exposure may have been multiple, i.e. the antigen may have been administered more than once over a period of time, e.g. hours, days, weeks or months. However, in a preferred embodiment of the invention, only a single previous exposure has occurred.

The nucleic acid construct may be DNA, RNA or cDNA capable of encoding a polypeptide comprising the antigen of interest. The polypeptide may comprise one or more antigens, i.e. a plurality of antigens, particularly two or more.

The nucleic acid construct may also encode a further immunomodulatory polypeptide. Preferably, this further/second immunomodulatory polypeptide will act as an adjuvant for protective immunity against antigen. Examples of further immunomodulatory polypeptides include immunogenic pathogen derived sequences, e.g. from tetanus toxoid fragment C (FrC) or a component therefore (e.g. DOM), plant viral coat proteins, e.g. potato Virus X coat protein (PVXCP), cytokine, Beta defensins (Biragyn A. 1999 Nature Biotechnology Vol. 17 p253-p258) and C3d complement system (Ross T. M. Nature Immunol. (1) p. 127-131, 2000). Other immunomodulatory polypeptides are described in Stevenson et al Immunological Review 2004, 199, p. 156-180 (incorporated by reference).

In a preferred embodiment of the invention, the immunomodulatory polypeptide is tetanus toxoid fragment C (FrC) or a component therefore, preferable DOM component. The inventors have shown herein and previously that p.DOM-antigen vaccine design can induce disease specific antibody response (WO 01/79510—U.S. Ser. No. 10/257,657—incorporated herein by reference).

Preferably, the nucleic acid construct is delivered by electroporation in unencapsidated form (i.e. not enclosed within a viral particle or other package). The nucleic acid may, however be associated with the external surface of a package or particle (e.g. a liposome).

The antigen is preferably derived from a pathogen, such as a virus (e.g. HSV, HIV, influenza virus, Haemophilus Influenzae etc); a bacteria (e.g. Staphylococcus, Salmonella, Meningococcus, mycobacteria, Pneumococcus etc) or from a parasite, e.g. malaria. However, in a preferred embodiment of the present invention, the antigen is a self or altered self-polypeptide or is derived from a self or an altered self-polypeptide. The self or altered self-polypeptide may be associated with an autoimmune disease (e.g. rheumatoid arthritis, multiple sclerosis, diabetes etc) or a cancer type. Most preferably the antigen is a tumor associated antigen or tumor specific antigen; mutated oncogenes or other self polypeptides displayed on the surface of tumors, or intracellular tumor polypeptides and oncofoetal antigens.

Ideally the expression of the nucleic acid construct in vivo should produce an antigen capable of stimulating antigen-specific B cells, cytotoxic T lymphocytes (CTLs), and helper T cells. In a preferred embodiment of the invention, the antigen is capable of inducing a CD4⁺ T-cell response or a CD8⁺ T-cell response, but most preferably, an antibody response. Of course, the method of the invention may induce more than one of these responses. In some embodiments, a method employing a Frc-antigen nucleic acid vaccine may stimulate CD4+ helper cells and antibody, and a method employing a p.DOM-antigen nucleic acid construct may induce CD8+ T cells (CTL).

The method may result in a peptide-specific cytotoxic response against cells expressing the antigen, e.g., against a cancer cell expressing a tumor associated antigen or tumor specific antigen. In some embodiments, the method may result in the killing of cancer cells without substantial killing of and/or substantial automimmune injury to non-cancer cells.

The inventors have surprisingly found that administration by electroporation of a nucleic acid construct for the purpose of boosting an already primed immune response against an antigen induced unexpectedly high levels of antibody, especially when priming with DNA alone. Electroporation at both time points (priming and boosting) is superior to no electroporation, but electroporation at the time of boosting only, is clearly most effective combination.

Therefore, in a preferred embodiment of the invention, there is provide a prime boost method of inducing an immune response, preferably an antibody response, to an antigen in an individual comprising the steps of firstly administering to said individual said antigen by a non-electroporation method; and secondly administering to said individual said antigen by electroporation; wherein the antigen administered second is in the form of a nucleic acid construct capable of encoding the antigen in vivo.

The first administration may be considered a priming antigen and the second administration may be considered a boosting antigen.

The antigen to be administered first (priming antigen) may be in the form of a nucleic acid construct encoding it, or it may be a polypeptide comprising it. The antigen may form part of a viral vector, be coupled to a cell or be encapsidated e.g. with liposomes. The nucleic acid construct may encode an immunomodulatory polypeptide as described above, as well as the priming antigen.

Non-electroporation methods of introducing the nucleic acid constructs into living cells in vivo are well known in the art. Conveniently, the nucleic acid is simply injected as naked DNA into the patient, e.g. intramuscularly, as a mixture with a physiologically acceptable diluent, such as a saline solution. Details of other methods and preferred embodiments of administration are described in U.S. Pat. Nos. 5,580,859 and 5,589,466. More involved methods of gene transfer include the use of viral vectors, encapsulating the DNA into liposomes, coupling of the DNA to cationic liposomes or to the outside of viruses (for review see Miller 1992, Nature 357, 45-46). These had the advantage of increased efficiency of transfer but, by comparison with direct injection of purified plasmid DNA, these alternative approaches are involved and can raise safety issues.

Electroporation is a well known technique for introducing nucleic acid into living cells. It is a method of transforming DNA, in which high voltage pulses of electricity are used to open pores in cell membranes, through which the foreign DNA can pass. For a review, see Tsong—Biophys. J. Vol. 60 1991 297-306.

In a second aspect of the invention, there is provided a method treating and/or preventing cancer (i.e. tumor growth) in an individual by inducing an immune response to a tumor, said method comprising first administering a priming tumor antigen to said individual; and secondly administering a boost tumor antigen to said individual by electroporation, wherein said boosting tumor antigen is in the form of a nucleic acid construct capable of expressing said antigen in vivo.

The tumor antigen is preferably derived from said individual, and may be a tumor specific antigen or a tumor associated antigen.

The priming antigen may be as described above, e.g., in the form of a nucleic acid construct or in the form of a polypeptide comprising said tumour antigen. Optionally, the priming tumour antigen is administered by intramuscular injection.

The tumour antigen may be capable of inducing an immune response selected from the group consisting of a CD4+ T-cell response, a CD8+ T-cell response, a cytotoxic T lymphocyte (CTL) response and an antibody response. The nucleic acid construct may be DNA, RNA or cDNA. The construct may comprise said antigen and a further immunomodulatory polypeptide, as described above.

The above method may be considered a method of vaccinating an individual against cancer where the boosting tumor associated antigen is administered repeatedly over several weeks, months or years to said individual. Generally, the methods of the invention may comprise one or more further boosting steps in addition to the administration of a boost tumour antigen to the individual with electroporation: these further steps may administer the antigen in the form of a polypeptide comprising the antigen or in the form of a nucleic acid construct encoding the antigen. One or more further boosting steps may comprise administration of a nucleic acid construct with electroporation.

The invention also provides a method of boosting an immune response in an individual to a tumor antigen, said individual having been previously primed against or exposed to said tumor antigen, said method comprising administering to said individual a nucleic acid construct encoding said tumor antigen by electroporation.

The individual may have been previously exposed to the tumor antigen by virtue of the presence of the tumor in the individual. Alternatively, the individual may have been previously administered with the tumor antigen. The previous administration of the antigen may have been via a nucleic acid construct or a polypeptide, or by a tumor cell and adjuvant directly. Other means of administering a tumor antigen are known to the skilled person. The nucleic acid construct used in the previous administration may optionally also encode an immunomodulatory polypeptide as described above.

The present inventors have exemplified the invention using tumor antigens from colon cancer, B-cell lymphoma and leukaemia. However, the skilled person will appreciate that the invention may be easily applied to other tumor associated antigens e.g. CEA, PSA PSMA etc, and other tumor specific antigens e.g. BCR-ABL. Ras etc.

Further, it is within the capabilities of the skilled person to obtain further tumor associated antigens for use in such methods preferably from an individual with a tumor already present.

The individual in question is preferably a human. However, the invention is also particularly applicable to other large mammals including cattle, horse, dogs, pigs, sheep, cats, monkeys, etc.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

FIG. 1. Suboptimal immunization conditions preclude effective priming of CD8⁺ T cells by DNA vaccination. Mice were vaccinated with p.DOM-AH1 or p.DOM DNA vaccines, as indicated, into the quadriceps muscle of each hind limb. Vaccines were delivered using either a constant injection volume (50 μl/leg) with varying DNA dose (a and b), or a constant DNA dose (25 μg/leg) with varying injection volume (c and d). At day 14, splenocytes from groups of three mice were pooled, and the numbers of AH1-specific CD8⁺ T cells producing IFN-γ were assessed ex vivo by intracellular labeling (a and c). Data from two experiments (Expts. 1 and 2) are shown in each panel. Splenocytes were also cultured with 1 μM AH1 peptide (b and d) for 6 days in vitro, before measuring CTL activity by ⁵¹Cr release assay. Targets were BCL1 cells, either alone (▴) or pulsed with AH1 peptide (•). Representative data from experiment 1 are shown in each case (b and d).

FIG. 2. Electroporation can rescue priming of CD8⁺ T cells following DNA vaccination using a suboptimal injection volume. Mice were vaccinated with p.DOM-AH1, either alone or in combination with electroporation (+EP), or with the control vaccine p.DOM combined with electroporation (p.DOM+EP). Mice received 25 μg of DNA vaccine per hind leg in an injection volume of 50, 25, or 10 μl (a, b, and c, respectively). At day 14, splenocytes were harvested, and the numbers of AH 1-specific CD8⁺ T cells producing IFN-γ were assessed ex vivo by intracellular labeling. Each point indicates a value from an individual mouse. Data combined from three of three (a) or two of two (b and c) identical experiments are shown in each panel. Data from each experiment are indicated using separate symbols (•, Expt. 1; ▴, Expt. 2; ▪, Expt. 3), with group means represented by a horizontal bar. Significant effects of electroporation were evident only using the lower injection volumes (b and c).

FIG. 3. The induction of protective immunity against CT26 tumor challenge is ablated by suboptimal DNA vaccine delivery, but is restored if combined with electroporation. Mice were vaccinated with p.DOM-AH1 or p.DOM DNA vaccines (25 μg/hind quadriceps muscle) using either an optimal or suboptimal injection volume (50 or 10 μl/leg, respectively) as indicated. Vaccines were administered alone or in combination with electroporation (+EP). At day 14 following vaccination, 1×10⁵ CT26 tumor cells were injected s.c., and mice were sacrificed when tumor size reached 15 mm diameter. Representative data from one of three similar experiments are shown.

FIG. 4. Therapeutic protection from pre-existing CT26 tumor can be recovered by combining suboptimal DNA vaccine delivery with electroporation. Mice were injected s.c. with 1×10⁵ CT26 tumor cells. One day later, tumor-bearing mice were vaccinated with p.DOM-AH1 or p.DOM DNA vaccines (25 μg/hind quadriceps muscle) using either an optimal or suboptimal injection volume (50 or 10 μl/leg, respectively), as indicated. Vaccines were administered alone or in combination with electroporation (+EP). Mice were sacrificed when tumor size reached 15 mm diameter. Data combined from three similar experiments are shown.

FIG. 5. Combining DNA vaccination and electroporation in a prime/boost regimen can increase CD8+ T cell responses. Mice were vaccinated with p.DOM-AH1 or p.DOM DNA vaccines using optimal conditions for AH1-specific CD8+ T cell priming (25 μg in 50 μl of saline per rear quadriceps muscle). Vaccines were administered alone or in combination with electroporation (+EP). Mice received booster injections of DNA vaccines at day 28, administered alone or with electroporation. At day 36, splenocytes were harvested, and the numbers of AH1-specific CD8⁺ T cells producing IFN-γ were assessed ex vivo by intracellular labelling. Each marker indicates data from an individual mouse, with group means represented by a horizontal bar. Data are combined from five of five identical experiments, each showing similar results.

FIG. 6. Improved anti-FrC and anti-BCL₁ IgG serum titers following DNA vaccination with electroporation. Mice were vaccinated with p.BCL1-FrC DNA vaccine (indicated as DNA), administered alone or in combination with electroporation (indicated as DNA+EP). Serum samples were collected at days 28 and 42, and the titers of anti-BCL₁ IgG (a) and anti-FrC IgG (b) were determined by ELISA. Each marker indicates data from an individual mouse, with median values represented by a horizontal bar. Background IgG levels to either Ag in mice receiving control vaccine (pcDNA3 without BCL₁-FrC insert) were below detectable limits (<10 U/ml). Data are combined from two of two identical experiments showing similar results.

FIG. 7. Combining DNA vaccination and electroporation in a sequential prime/boost regimen can generate superior humoral responses. Mice were vaccinated with p.BCL1-FrC DNA vaccine (indicated as DNA), administered alone or in combination with electroporation (indicated as DNA+EP). Additional booster vaccinations were given at day 21, with or without electroporation. Serum samples were collected at day 41, and the titers of anti-BCL₁ IgG (a) and anti-FrC IgG (b) were determined by ELISA. Each marker indicates data from an individual mouse, with median values represented by a horizontal bar. Background IgG levels to either Ag in mice receiving control vaccine (pcDNA3 without BCL₁-FrC insert) were below detectable limits (<10 U/ml). Data are combined from two of two identical experiments showing similar results.

FIG. 8. exemplifies induction of disease-specific CD4+ T cells using p.DOM-peptide DNA fusion vaccine design.

FIG. 9. exemplifies induction of disease-specific antibody response using p.DOM-antigen fusion vaccine design.

FIG. 10. Schematic diagram indicating DNA fusion vaccine design. The control vaccine p.DOM contains sequence encoding the amino terminal domain (DOM) of Fragment C from tetanus toxin, including the p30 CD4⁺ Th epitope p.DOM-gag includes DNA sequence encoding the immunodominant H-2 D^{b}-restricted gag₈₅₋₉₃ CTL epitope derived from FMuLV_{gag} linked to the C-terminus of DOM. Each vaccine includes a leader sequence at the amino terminus. Vaccine sequences were assembled and inserted into the commercial vector pcDNA3 using Hind III and Not I restriction sites.

FIG. 11. Ex vivo detection of gag-specific T cells in wt and gag-Tg mice following DNA vaccination. Wt (A) and gag-Tg (B) mice were immunized with p.DOM-gag or p.DOM DNA vaccines, or irradiated FBL-3 cells, as indicated, on days 0 and 28; DNA booster injections at day 28 were administered with electroporation. Splenocytes were harvested from individual mice at day 36 and the numbers of spot forming cells (SFC) secreting IFNγ were assessed ex vivo by ELISpot assay. (A, B) Splenocytes were incubated with gag₈₅₋₉₃ peptide to assess the epitope-specific CD8⁺ T cell response and the p30 peptide to assess CD4⁺ T cell responses to the DOM component of the vaccine. Test samples were set up in triplicate; baseline responses without peptides are indicated. The data indicate mean SFC/million splenocytes and the standard error of the mean (sem; error bars). Representative data from 1 of 2 identical experiments are shown. (C) The mean number of gag₈₅₋₉₃ epitope-specific SFC was compared between wt and gag-Tg mice. Responses to 1 μM gag₈₅₋₉₃ peptide were pooled from individual mice; data are expressed as mean SFC/million splenocytes, together with the sem. The number (n) of animals pooled per group is indicated.

FIG. 12. Ex vivo detection of gag-specific CD8⁺ T cells in both wt and gag-Tg mice following DNA vaccination. Wt and gag-Tg mice were immunized with p.DOM-gag or p.DOM DNA vaccines, or irradiated FBL-3 cells, as indicated, on days 0 and 28; DNA booster injections at day 28 were administered with electroporation. At day 36, splenocytes were harvested and the frequency of gag₈₅₋₉₃-specific CD8⁺ T cells producing IFNγ was assessed ex vivo by intracellular cytokine staining. (A) Representative data from individual mice. (B) Data pooled from individual mice indicating the mean percentage of CD8⁺ T cells producing IFNγ in response to gag₈₅₋₉₃ peptide (error bars: standard error of the mean).

FIG. 13. DNA vaccination induces lower avidity gag₈₅₋₉₃-specific CTL in gag-Tg mice compared to wt mice. Wt and gag-Tg mice were immunized with p.DOM-gag on days 0 and 28; booster injections at day 28 were administered with electroporation. Splenocytes were harvested from individual mice at day 36 and the numbers of spot forming cells (SFC) secreting IFNγ were assessed ex vivo by ELISpot assay. Splenocytes were incubated with varying concentrations of gag₈₅₋₉₃ peptide to assess the epitope-specific CD8⁺ T cell response; test samples were set up in triplicate for each peptide concentration. Baseline responses without peptide were subtracted and the number of SFC/million splenocytes was calculated as a percentage of the maximum number of SFC/million splenocytes responding per mouse; data were then pooled for each experimental group to calculate the mean ELISpot response, together with the standard error of the mean (error bars). Representative data from one of 2 identical experiments are shown.

FIG. 14 Absence of autoimmune liver injury following DNA vaccination of gag-Tg mice. Gag-Tg mice were immunized with p.DOM-gag on days 0 and 28; DNA booster injections at day 28 were administered with electroporation. Unimmunized mice served as naïve controls. Mice were euthanized at day 36 and liver samples were fixed in formaldehyde, paraffin-embedded, sectioned and stained with hematoxylin/eosin. Coded specimens were analyzed for inflammation and lymphocyte infiltration by a liver pathologist in a blinded manner. All portal tracts were normal, with no expansion and normal biliary structures. There was no inflammation in portal tracts or parenchyma. There was no hepatocyte degeneration or loss, and the normal sinusoidal architecture was present. Representative sections from gag-Tg mice immunized with p.DOM-gag (A) or naïve gag-Tg controls (B) are shown (magnification, ×10).

FIG. 15. DNA vaccination of gag-Tg mice induces gag₈₅₋₉₃-specific effector CTL able to lyse FBL-3 leukemia cells in vitro. Wt and gag-Tg mice were immunized with p.DOM-gag or p.DOM DNA vaccines on days 0 and 28; booster injections at day 28 were administered with electroporation. At day 36, splenocytes from individual mice were cultured with 0.01 μM gag₈₅₋₉₃ peptide for 6 days in vitro, prior to measuring CTL activity by standard ⁵¹Cr-release assay. Targets were EL4 cells pulsed with gag₈₅₋₉₃ peptide or an H-2 D^{b}-restricted control peptide (Uty), FBL-3 leukemia cells expressing endogenous FMuLV_{gag} antigen or the NK-susceptible control cell line YAC-1, as indicated. No lytic activity was detectable in splenocyte cultures from mice vaccinated with the control vaccine (p.DOM). Representative data from individual mice are shown; data from one of two identical experiments are shown. Error bars: standard error of the mean.

FIG. 16. DNA vaccination of gag-Tg mice induces gag₈₅₋₉₃-specific effector CTL capable of in vivo cytolytic activity. Wt and gag-Tg mice were immunized with p.DOM-gag or p.DOM DNA vaccines, or irradiated FBL-3 cells, as indicated, on days 0 and 28; DNA booster injections at day 28 were administered with electroporation. At day 36 mice were infused, by intravenous injection, with sex-matched syngeneic splenocytes that had been pulsed with gag₈₅₋₉₃ (gag) peptide or control (con) peptide (SNWYFNHL) and labeled with different concentrations of CFSE (gag₈₅₋₉₃ peptide: CFSE^{hi}; control peptide: CFSE^{low}). Splenocytes were harvested 20 hours later and the frequency of CFSE-labeled target cells was determined by FACS analysis. (A) Representative FACS profiles from individual mice. (B) The ratio of gag₈₅₋₉₃ to control peptide-pulsed cells surviving in each recipient was calculated. The ratios were normalized, by assuming that the mean ratio was 1:1 in mice given the control vaccine (p.DOM), and pooled to calculate the mean proportion of surviving donor cells pulsed with the gag₈₅₋₉₃ peptide, together with the standard error of the mean. Data are pooled from two of two identical experiments, each giving similar results; the number (n) of animals pooled per group is indicated.

FIG. 17. DNA vaccination of gag-Tg mice induces gag₈₅₋₉₃-specific effector CTL able to protect mice from FBL-3 leukemia. Wt (A) and gag-Tg (B) mice were immunized with p.DOM-gag or p.DOM DNA vaccines as indicated, on days 0 and 28; DNA booster injections at day 28 were administered with electroporation. Naïve mice served as controls. At day 36 mice were challenged by i.p. injection of FBL-3 leukemia cells and tumor development was monitored. Survival data in each panel (A, B) are pooled from two of two identical experiments, each giving similar results.

### DETAILED DESCRIPTION

### Example 1

Materials and Methods

Cells

The murine CT26 colon carcinoma cell line and a cell line derived from the B cell lymphoma BCL₁ (36) were maintained in RPMI 1640 medium supplemented with 10% heat-inactivated FCS (Invitrogen Life Technologies), 1 mM sodium pyruvate, 2 mM L-glutamine, nonessential amino acids (1% of 100× stock), 25 mM HEPES buffer, and 50 μM 2-ME (hereafter referred to as complete medium). CT26 cells were harvested by incubation with Ca/Mg-free medium, as previously described (23).

Peptides

The H-2L^{d}-restricted gp70 epitope (AH1) has been described previously (35). The peptide (SPSYVYHQF) was synthesized commercially and supplied at >95% purity (Peptide Protein Research). Peptide stocks (2 mM) were dissolved in PBS, filter sterilized, and stored at −20° C.

DNA Vaccines

Construction of the DNA fusion vaccine p.DOM-AH1 has been described (23). It encodes the first domain of fragment C (FrC)³ from tetanus toxin (DOM; TT₈₆₅₋₁₁₂₀) with sequence encoding the AH1 CTL epitope fused to the 3′ terminus. The p.DOM control vaccine encodes the first domain of FrC alone.

p.BCL₁, encoding the idiotypic V_{L} and V_{H} regions (single chain Fv (scFv)) derived from the murine B cell lymphoma, BCL₁, fused to human CH₃ from I_{g}G1, has been described previously (37). This was used as a template to construct p.BCL₁-FrC (kindly supplied by D. Zhu University of Southampton, Southhampton, U.K.), which encodes BCL₁ scFv upstream of sequence-encoding FrC. Briefly, C_{H}3-encoding sequence was cut from p.BCL₁ using BspEI and NotI. FrC sequence (8) was amplified using the primers 5′-TATTCCGGAGGACCCGGACCTATGAAA-3′ (forward) and 5′-TAATGCGGCCGCTTAGTCGTTGGTCCAACCTTC-3′ (reverse), each of which introduced either a BspEI site or NotI site, respectively, to the FrC termini. The resulting PCR product was gel purified, digested, and cloned into p.BCL₁ in the place of CH₃, creating p.BCL₁-FrC, which encodes signal peptide-V_{L}-linker peptide-V_{H}-linker peptide-FrC.

Each DNA vaccine encoded the signal sequence derived from the V_{H} of the IgM of the BCL₁ tumor, and was incorporated into the pcDNA3 vector backbone (Invitrogen Life Technologies). Vaccine integrity was confirmed by DNA sequencing, while expression and product size were checked in vitro using the TNT T7 Coupled Reticulocyte Lysate System (Promega).

Vaccination Protocol

BALB/c (H-2^{d}) mice were vaccinated at 6-12 wk of age by injection of DNA, in 0.9% saline (w/v), into the quadriceps muscle of each hind limb. Injection volume per leg and total DNA dose are indicated, but ranged from 10 to 50 μl/leg and 5 to 100 μg of DNA/leg (10-200 μg dose). A Hamilton Microliter syringe (Scientific Laboratory Supplies) was used to administer injection volumes smaller than 50 μl. All injections were administered using a 26 G needle. Animal welfare and experimentation were conducted in accordance with local Ethical Committee and United Kingdom Coordinating Committee for Cancer Research guidelines, under Home Office license.

Electroporation In Vivo

Mice were anesthetized before electroporation using 1 part midazolam (5 mg/ml), 1 part hypnorm (fentanyl citrate (0.315 mg/ml) and fuanisone (10 mg/ml)), and 2 parts water. The mice received 7 μl/g body weight by i.p. injection. The skin overlying the quadriceps muscle was shaved, and DNA vaccine was administered using the indicated dose and volume. Following the application of a conductance gel, silver electrodes were placed on the skin on either side of the injection site and a local electrical field was immediately applied using a custom-made pulse generator, Elgen (Inovio), as previously described (31). The electrical field comprised 10 trains of 1000 square wave pulses delivered at a frequency of 1000 Hz, with each pulse lasting a total of 400 μs (200 μs positive and 200 μs negative). The electrical field strength varied with the resistance in the tissue of each animal and was ˜50 V over 3-4 mm. Each train was delivered at 1-s intervals; the electrical pulse was kept constant at ±50 mAmp (31).

Ex Vivo Intracellular IFN-γ Assay

To assess priming of CD8⁺ T cells, mice were culled at day 14 following DNA vaccination (using the dose and volume of vaccine, as indicated), and spleens were harvested and processed for detection of intracellular IFN-γ. To monitor the potential to boost existing CD8⁺ T cell responses, mice were vaccinated at day 0 (25 μg of DNA in 50 μl of saline per rear limb) and given booster injections of vaccine at day 28, either with or without electroporation at each time point; spleens were harvested at day 36 to monitor CD8⁺ T cell responses. Viable, pooled splenocytes were selected by density centrifugation, and cells were incubated for 4 h at 37° C. in 96-well plates, at 1×10⁶ cells/well, in complete medium together with 10 U/well human rIL-2 (PerkinElmer), 1 μM AH1 peptide, and 1 μl/well Golgi Plug. Samples were then processed to label intracellular IFN-γ, as previously described (23), before analysis by FACSCalibur using CellQuest software (BD Biosciences). Analyses were performed on lymphocyte populations with MHC class II-positive cells gated out.

CTL Assay

Mice were culled at day 14 postvaccination, spleens were pooled, and splenocyte suspensions (3×10⁶ cells/ml) were prepared in complete medium, together with IL-2 (20 U/ml) and AH1 peptide (1 μM). Bulk splenocyte cultures were incubated at 37° C., 5% CO₂, for 6 days before assessing cytolytic activity in a standard 4- to 6-h ⁵¹Cr release assay, as previously described (23). Targets included BCL₁ cells, either alone or labeled with AH1 peptide. Specific lysis was calculated by the standard formula ((release by CTL−spontaneous release)/(total release−spontaneous release)×100%). Spontaneous release was always <30%.

Tumor Challenge

Mice were vaccinated with a total dose of 50 μg of DNA (25 μg per rear leg), using the indicated injection volumes administered with or without electroporation. During tumor challenge, mice were injected s.c. with 1×10⁵ CT26 tumor cells into the rear flank. For prophylacetic immunization, mice were challenged with tumor cells 14 days after DNA vaccination, while for therapeutic immunization tumor cells were injected 1 day before DNA vaccination. All mice were monitored twice daily for tumor development and were culled when mean tumor diameter reached 15 mm, in accordance with humane end point guidelines (United Kingdom Coordinating Committee for Cancer Research).

Assessment of Ab Titers

To monitor priming of humoral immunity, mice were vaccinated i.m. with 50 μg of p.BCL₁-FrC (25 μg in 50 μl of saline per rear limb) on day 0, either with or without electroporation. Serum samples were collected on days 28 and 42 and analyzed by ELISA for the presence of IgG specific for BCL₁ Id IgM or FrC, as described previously (8, 38, 39). For the prime/boost setting, the inventors vaccinated mice at day 0 and gave booster vaccinations at day 21, with or without electroporation at each time point, and collected serum samples at day 41 to monitor Ab responses. The injection schedule (day 0, day 21) follows the previously published protocol for Ab induction, established using Id IgM protein vaccination (34). For protein vaccinations, Id IgM from BCL₁ was coupled to FrC protein using a one-step glutaraldehyde method, as used for coupling to keyhole limpet hemocyanin (38). Mice were injected with IgM, or IgM coupled to FrC, in CFA before serum analysis, as described previously (34, 38). ELISA plates were analyzed using a Dynex MRX plate reader at 450 nM wavelength.

Statistical Analysis

CTL responses were analyzed using the Mann-Whitney U test. Serum IgG titers were compared using a two-tailed t test on log normalized data. Survival curves were compared using the χ² log rank test. Experimental groups were considered significantly different from control groups if p<0.05.

Effect of DNA Vaccine Dose on Induction of AH1-Specific CD8⁺ CTL

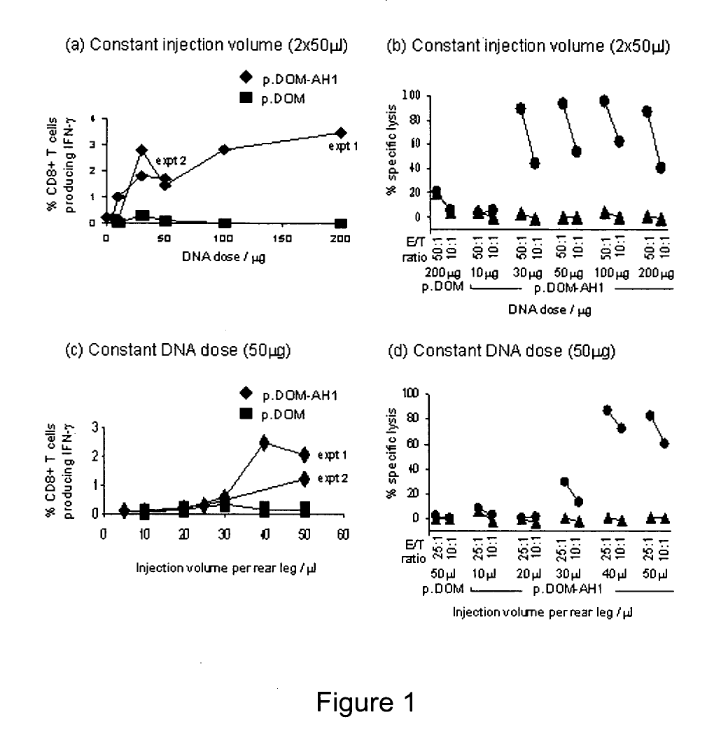

The inventors tested the ability of the p.DOM-AH1 vaccine to induce AH1-specific CD8⁺ T cell responses when the vaccine dose was varied (FIG. 1, a and b). Using a constant injection volume of 2×50 μl, a dose of 10 μg was inadequate to induce a detectable CD8⁺ T cell response. Increasing the dose to 30 μg induced significant IFN-γ-producing CD8⁺ T cell responses, and this was not amplified markedly by using 50-200 μg doses (FIG. 1a, Expt. 1). A second experiment confirmed this trend (FIG. 1a, Expt. 2); an AH1-specific CD8⁺ T cell response was only detected when a dose of >5 μg was used, with the proportion of responding cells reaching a plateau at a dose of 30 μg of p.DOM-AH1. Responses to the control vaccine (p.DOM) were insignificant. The functional efficacy of the IFN-γ-producing CD8⁺ T cells was confirmed in a cytotoxicity assay following a 6-day expansion in vitro with AH1 peptide (FIG. 1b). The CTL specifically lysed BCL₁ cells when pulsed with peptide (FIG. 1b), as well as tumor cells expressing endogenous AH1 (23) (data not shown).

Effect of DNA Vaccine Injection Volume on Induction of AH1-Specific CD8⁺ CTL

Maintaining a constant DNA vaccine dose of 50 μg per mouse, the inventors then assessed the impact of injection volume on AH1-specific CD8⁺ T cell induction (FIG. 1, c and d). It was necessary to use 30 μl per limb to generate a significant response, and 40 μl for maximum response, not increased further using 50 μl (FIG. 1c, Expt. 1). The effect of injection volume on immune outcome was confirmed in a second experiment (FIG. 1c, Expt. 2); an injection volume of 25 μl was required to prime a significant AH1-specific CD8⁺ T cell response, with 50 μl producing an increased response. Responses to the control vaccine (p.DOM) were insignificant. Again, functional activity was confirmed by cytotoxicity assay following a 6-day expansion in vitro (FIG. 1d).

Electroporation can Enhance CD8⁺ T Cell Induction Following Suboptimal Vaccine Delivery

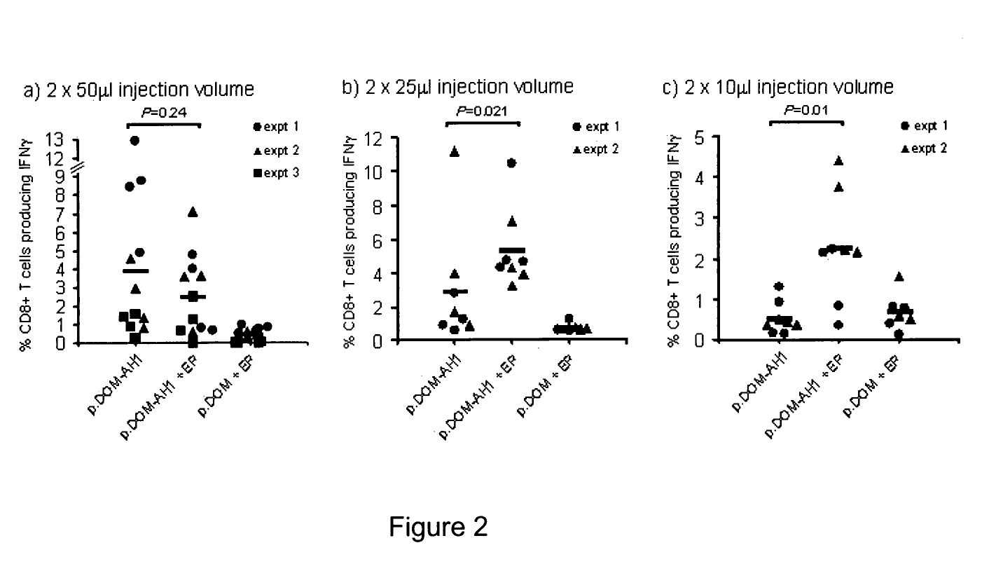

The inventors next assessed the effects of combining DNA vaccine delivery with electroporation. Mice were vaccinated with 50 μg of p.DOMAH1 using injection volumes that were either optimal (2×50 μl) or suboptimal (2×25 μl and 2×10 μl) for priming of AH1-specific CD8⁺ T cells (FIG. 1c), with electroporation of the injection sites. Results (FIG. 2) indicate that an injection volume of 50 μl led to effective priming of AH1-specific CD8⁺ T cells, with decreasing responses as volumes were reduced (FIG. 2). Electroporation did not influence the performance of the 50 μl delivery (FIG. 2a). Three experiments were conducted to confirm this point, because experiment 1 showed a decrease in response when using electroporation. However, the additional two experiments showed no change, and data compiled from the three identical experiments indicated no effect of electroporation using this optimized volume (FIG. 2a). In contrast, electroporation did significantly improve CD8⁺ T cell responses when using suboptimal injection volumes of 25 and 10 μl per limb, p=0.021 and p=0.01, respectively, and the trend was evident in each of the two experiments (FIG. 2, b and c).

Rescued CTL Responses can Protect Against CT26 Tumor Cell Growth In Vivo

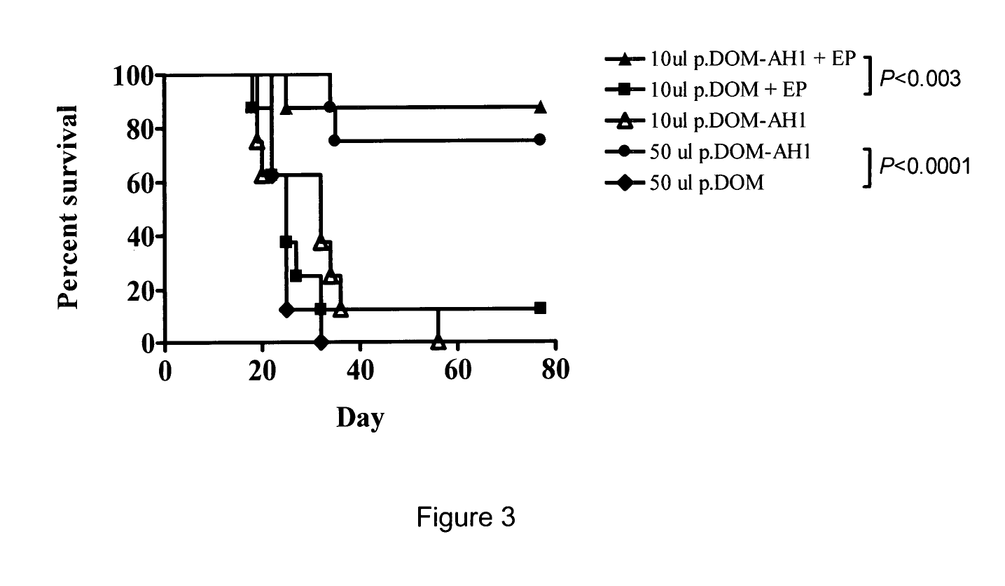

The inventors have previously demonstrated that following vaccination with p.DOM-AH1, the induced CD8⁺ CTL of single epitope specificity can protect against tumor (23—incorporated by reference). Results (FIG. 3) confirm this using their standard injection volume of 50 μl/leg, with tumor challenge 14 days later. Delivery in a suboptimal volume (2×10 μl) did not mediate protection. However, protective efficacy was completely restored when suboptimal volume was combined with electroporation (p<0.003), demonstrating a clear correlation between the ability to induce cytolytic T cells by vaccination and protection from CT26 tumor in vivo.

Therapeutic Protection from CT26 Using DNA Vaccination and Electroporation

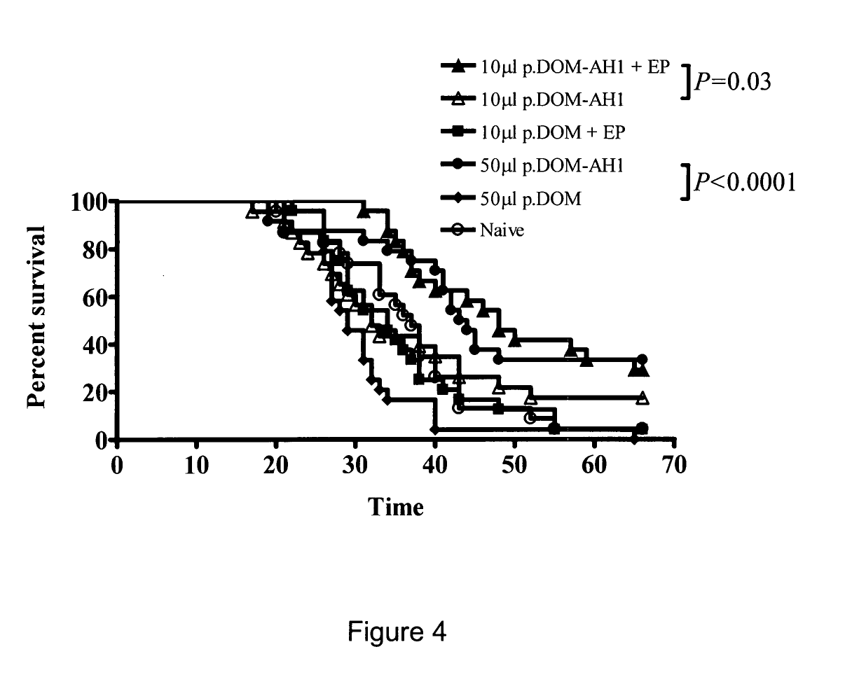

To assess therapeutic efficacy, the inventors investigated the effects Of DNA vaccination 1 day after tumor injection. Again, mice received a total dose of 50 μg of DNA, but the vaccine injection volume was varied. Results (FIG. 4) indicate that vaccination with p.DOM-AH1 using our standard injection volume (2×50 μl) activates protective immunity in that setting compared with non-vaccinated mice or those given the control vaccine (p.DOM). Delivery in a suboptimal injection volume (2×10 μl) was ineffective, but protection could be fully restored by combination with electroporation (p=0.03).

Electroporation in a Prime/Boost Regimen can Further Increase the Tumor-Specific CD8⁺ T Cell Response

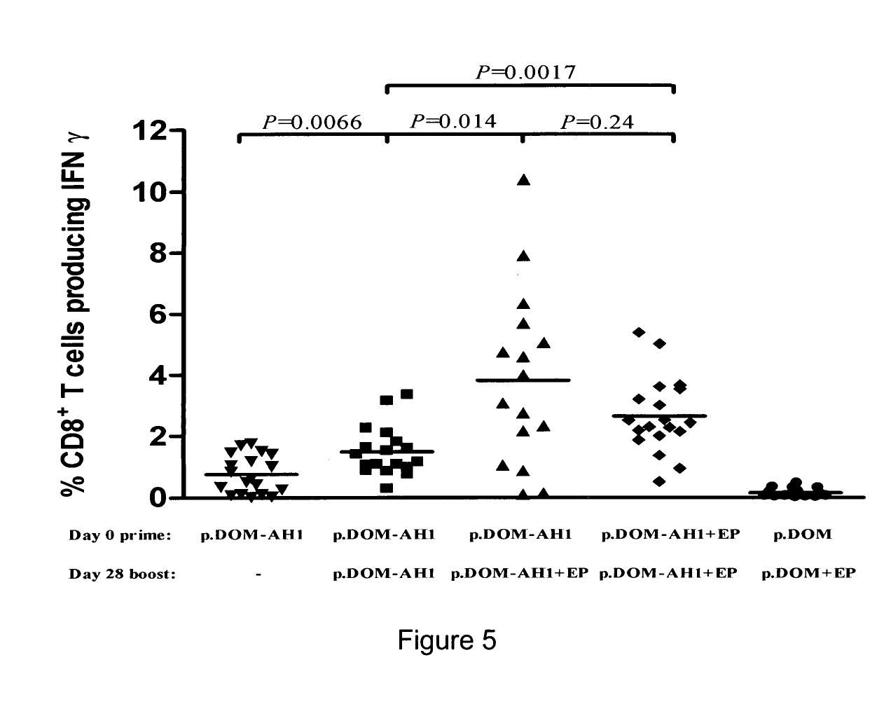

The effects of electroporation on priming were clear only when using suboptimal vaccination conditions. The inventors then investigated whether electroporation could improve performance of optimal delivery when combined with boosting. Electroporation was given either at priming alone, at boosting alone, or at both time points. Boosts were given at day 28, and the levels of AH1-specific IFN-γ-producing CD8⁺ T cells were measured ex vivo 8 days later (day 36) (FIG. 5), because previous data using this vaccine design indicated that peak CD8+ T cell responses occur 7-10 days postboost (40).

At this later time point after only the first injection, the proportions of detectable AH 1-specific CD8⁺ T cells observed in mice primed with p.DOM-AH1 at day 0 only (without electroporation) were low (mean 0.76%), even undetectable in five mice, probably due to the natural kinetics of the CD8⁺ T cell response. Booster injections at day 28 (without electroporation) generated a significant increase in the proportion of AH1-specific IFN-γ-positive CD8⁺ T cells, enabling them to be detected in all mice (mean 1.5%, p=0.0066). However, the application of electroporation at the time of boosting amplified this response, generating high levels of IFN-γ-positive CD8⁺ T cells (mean 3.8%, p=0.014) (FIG. 5). Electroporation at both the priming and boosting stages was also effective in amplifying the response to naked DNA (mean 2.7%, p=0.0017). There was a trend for double electroporation to be less effective than priming with DNA alone plus boosting with electroporation, but the difference was not statistically significant (p=0.24). Interestingly, reversing the prime/boost strategy (priming with DNA plus electroporation and boosting with DNA alone) did not enhance the number of responding T cells compared with DNA injection alone (data not shown), indicating that the correct sequence of vaccination and electroporation is critical for boosting the CD8⁺ T cell response.

Electroporation can Enhance Priming of the Antitumor IgG Response by DNA Vaccination

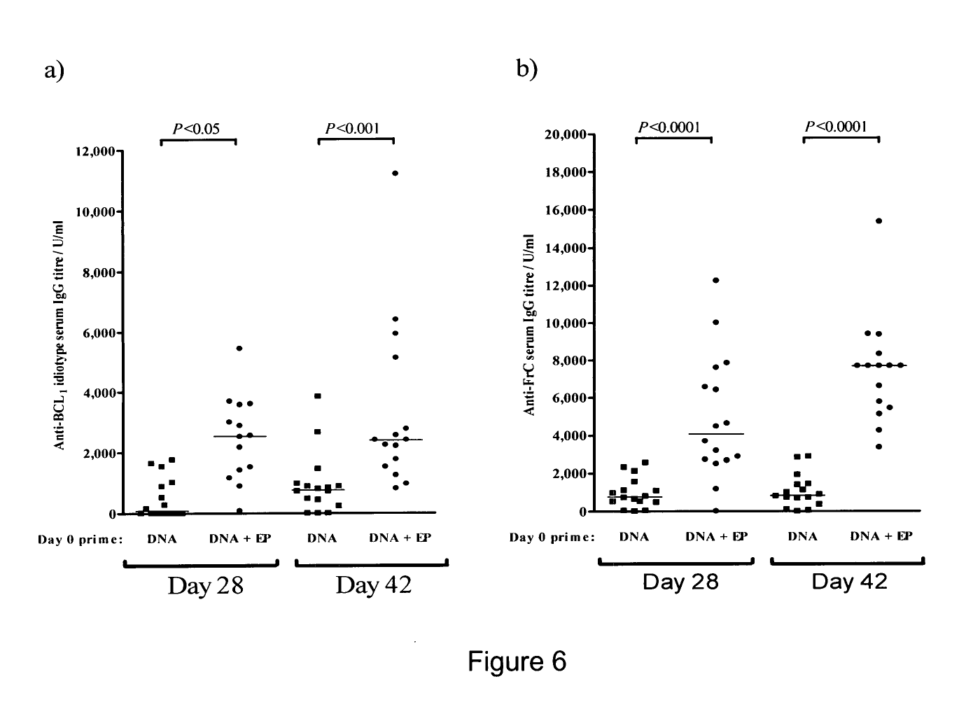

To measure the effect of electroporation on induction of Ab, the inventors used the DNA fusion vaccine containing the V regions of the BCL₁ lymphoma linked as scFv to full-length FrC (p.scFv-FrC) (8, 13). This vaccine is known to induce significant levels of Ab against both tumor-derived idiotypic Ig and FrC components of the fusion gene, and this is confirmed in FIG. 6. A single injection of the p.scFv-FrC vaccine induced detectable anti-Id Ab at day 28, which had increased only slightly by day 42. Electroporation at priming increased anti-Id Ab at both time points. Similar effects were evident in the anti-FrC Ab responses (FIG. 6b).

Electroporation in a Prime/Boost Regimen can Further Increase Ab Responses

The inventors then tested the effects of electroporation used at the stage of priming and/or boosting (day 21) on Ab responses to both Id and FrC measured at day 41.

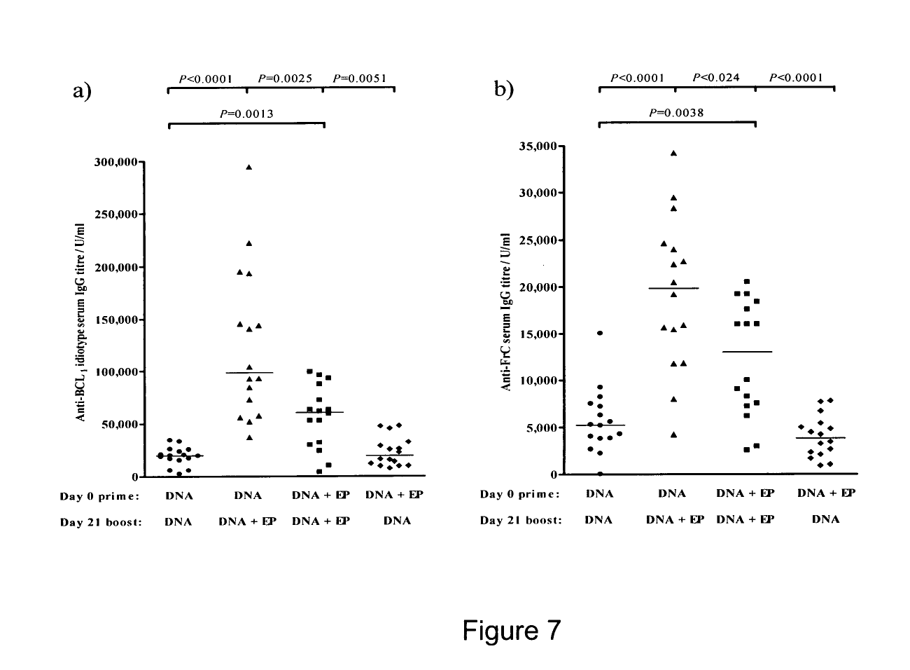

Priming and boosting with DNA alone induced significant levels of anti-Id IgG, detectable in all vaccinated mice (FIG. 7a). However, priming with DNA alone and boosting with DNA plus electroporation led to a striking amplification of the anti-Id response (p<0.0001). Electroporation at both time points is superior to no electroporation (p=0.0013), but electroporation at the time of boosting only is clearly the most effective combination (p=0.0025) (FIG. 7a). Interestingly, reversal of this prime/boost strategy (electroporation at the time of priming only) did not improve the anti-Id response by day 41, compared with DNA alone (p=0.40). Again, similar data were obtained for the anti-FrC response (FIG. 7b). The anti-Id levels achieved by adding electroporation to boosting are ˜7-fold higher than those achieved using DNA alone (FIG. 7a). These levels are similar to those induced by the previous gold standard of idiotypic protein plus CFA (34) and are comparable with the ˜11-fold increase over DNA scFv-FrC observed by using Id-FrC fusion protein in CFA (data not shown).

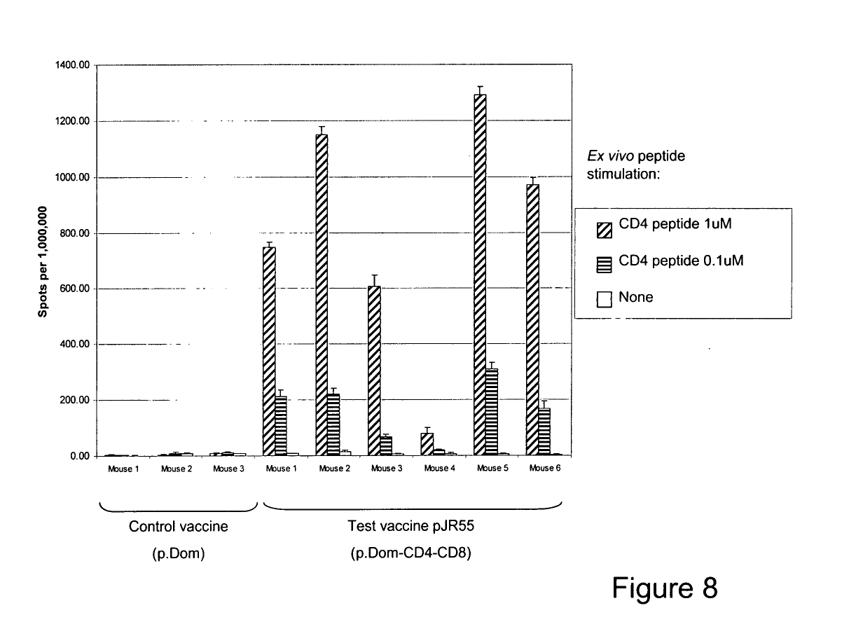

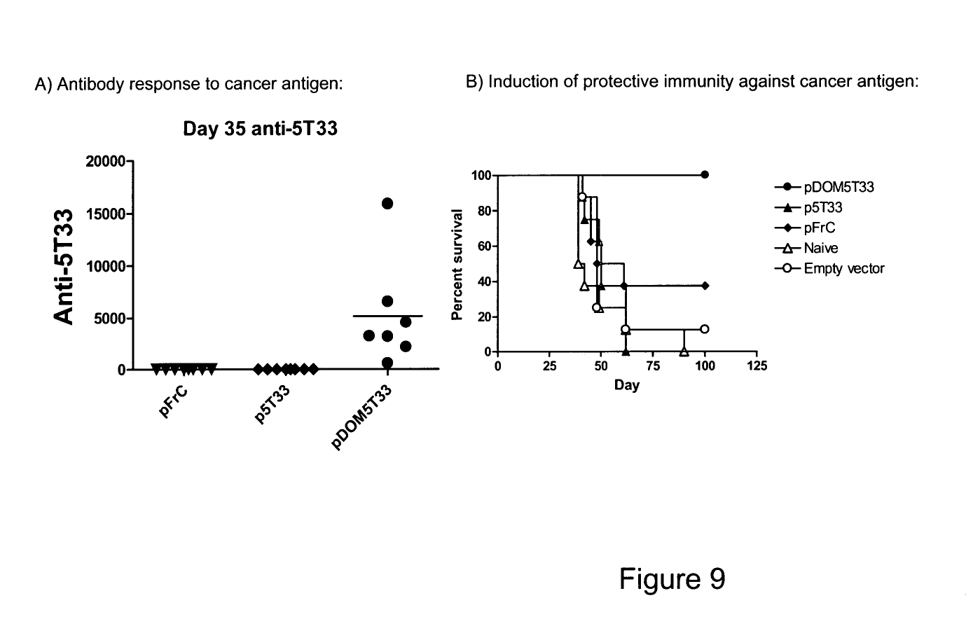

By way of exemplification, FIG. 8 and FIG. 9 show induction of a disease-specific CD4+ T cell and antibody response respectively, using p.DOM-antigen DNA fusion vaccine design.

FIG. 8 shows a DNA vaccine (pJR55) encoding Fragment C component (DOM) with disease-specific polypeptide sequence fused to the C terminus, this includes a CD4+ T cell motif from Mycobacterium tuberculosis antigen 85 A. Mice were vaccinated at days 0 and 28 with DNA vaccines (day 28 injections given with electroporation to aid delivery). The mice were then culled at day 36 and ex vivo CD4+ T cell responses assessed by ELISpot assay. The CD4+ T cell responses detected against both p.DOM vaccine component (data not shown) and Tuberculosis disease component (indicated in FIG. 8), demonstrate that p.DOM-antigen is from an infectious disease, demonstrating the applicability of this approach against both cancer antigens and other diseases.

FIG. 9 shows a DNA vaccine (pDOM-5T33) encoding a fragment C component (DOM) with disease-specific polypeptide sequence fused to the C-terminus. In this case, it is the idiotype scFv from 5T33 murine myeloma. Mice were vaccinated at days 0 and 21 and 42 with DNA vaccines. The mice were bled at day 35 to assess anti-5T33 myeloma cancer cells at day 63 and survival monitored. Anti-5T33 antibody responses were detected (A). Vaccine clearly protects from 5T33 cancer (B). 5T33 cells are idiotype negative at the surface so mechanism or protection is unclear. However, it is possibly through additional induction of anti-idiotypic CD4+ T cells. These results demonstrate that p.DOM-antigen vaccine design can induce disease-specific antibody response. In this case the target antigen if from a tumor cell.

There are two major problems in developing DNA vaccination as a treatment for cancer. The first is the poor immunogenicity of most candidate tumor Ags. There are many strategies aimed to increase this (20), and we have chosen to use fusion genes that encode tumor Ags in combination with immunogenic pathogen derived sequences, mainly derived from tetanus toxin (20). Different designs have been optimized to induce effector pathways for precision attack on tumor targets (21). Currently, the inventors are testing these in clinical trials, with early evidence for immune responses.

The second problem, relevant for all DNA vaccines, relates to the translation of promising data in animal models to human subjects. Although safety does not appear to be an issue, the efficacy in humans has been disappointing (41-43), partly due to difficulties in scaling up DNA vaccine dose and injection volume for human application (21).

Cellular uptake of DNA appears to be a significant limiting factor on transfection in vivo, and low vaccine dose results in poor Ag expression and reduced immunogenicity (27). Similarly, injection volume can influence Ag expression and immunogenicity in vivo (26). Hydrostatic pressure created by a relatively large injection volume into a small muscle may distend the extracellular space between muscle cells and facilitate the transfer of macromolecules across the plasma membrane (26). This effect will be reduced in large animals and humans, because the ratio of injection volume to muscle mass is far lower (26). In vivo electroporation can increase DNA uptake by muscle cells and mononuclear cells at the site of injection (27, 44), leading to increased Ag expression (29-31). Dendritic cells at the draining lymph nodes have been shown to contain DNA originating from the injection site (26), and electroporation might also contribute an undefined adjuvant effect, possibly mediated through local tissue damage and release of inflammatory factors (44-46).

The inventor's murine data confirm that induction of antitumor CTL by DNA fusion vaccines is dependent on dose and volume of injection (26, 27). It is clearly possible to achieve an optimal dose/volume in mice, and electroporation then has no additive effect on priming. However, induction of Ab appears far from optimal under the same conditions and electroporation amplifies priming significantly.

This could reflect a need for higher levels of Ag for priming of Ab responses (47, 48). Electroporation therefore offers a strategy to amplify priming, which could be useful in the clinic.

However, a more striking effect of electroporation was evident in a prime/boost setting, with naked DNA at both time points. The amplification is reminiscent of that achieved by boosting with Ags delivered via viral vectors (49). These vectors are presumed both to increase protein expression and to stimulate an inflammatory response (50, 51). Their disadvantages, particularly for cancer patients, are that pre-existing or developing immunity can neutralize the delivery agent and negate continued use (52-54). A more general disadvantage is that highly immunogenic viral or bacterial vectors may introduce potentially immunodominant T cell epitopes, possibly out-competing weakly immunogenic tumor Ags in the ensuing immune response (55-57). Efforts are being made to overcome these problems by removing viral genes (58), but success there may deplete efficacy, and two vaccine vehicles mean more safety/regulatory issues.

The mechanism by which electroporation amplifies CTL or Ab responses when administered at the stage of boosting is unclear. Increased Ag expression is likely to be important for boosting CTL, possibly by increasing the numbers of Ag-loaded APC. Our prime/boost strategy will drive increased Ag expression at the crucial stage of boosting, leading to more effective activation of vaccine-specific CD8⁺ T cells. Electroporation at both priming and boosting also enhanced CD8⁺ T cell induction, but was no more effective than using electroporation only at the boosting stage, confirming that the availability of Ag at boosting, rather than priming, is critical for CD8⁺ T cell induction. For Ab induction, in addition to a more effective induction of T cell help, more available Ag would be provided on boosting for uptake by B cells (47, 48, 59). This may explain why priming with DNA plus electroporation and boosting with DNA alone was no more effective at raising specific Ab levels than injecting DNA alone at both time points. Electroporation also leads to an inflammatory response, which is likely to recruit specific T and B cells to the injection site (44-46).

With this in mind, the inventors delineated a homologous prime/boost strategy in which mice received the same naked DNA fusion vaccine, with electroporation only at the critical time of boosting. This turned suboptimal delivery for CTL induction into effective vaccination and should be translatable to human subjects. Electroporation devices are now acceptable for human subjects (60) and have already been tested in volunteers (61). The inventors have started a clinical trial in patients using the same device (61). Protocols for electrical stimulation have to balance immune outcome with patient acceptability, and further trials in large animals and patients will assist optimization. The apparently suboptimal performance of DNA vaccines in inducing Ab responses can be improved by the same prime/boost strategy. The priming qualities of DNA vaccines, together with the improved Ag expression offered by electroporation, can now be combined in a homologous prime/boost approach to generate superior immune responses. This simple modification should facilitate application to the clinic.

### Example 2

Materials and Methods

Cells

FBL-3 is a Friend virus-induced erythroleukemia of C57BL/6 (B6) origin (H-2^{b}) which causes disseminated disease; it expresses FMuLV gag- and env-encoded products and MHC class I molecules⁶². EL4 is a chemically-induced T cell lymphoma derived from C57BL/6N mice, and YAC-1 is an NK-susceptible T cell lymphoma originating from the A/Sn strain. All cells were maintained in RPMI 1640 medium supplemented with 10% heat-inactivated FCS (Life Technologies, Paisley, UK), 1 mM sodium pyruvate, 2 mM L-glutamine, non-essential amino acids (1% of 100× stock), 25 mM HEPES buffer and 50 μM 2-mercaptoethanol (complete medium).

Peptides

The H-2 D^{b}-restricted gag peptide (gag₈₅₋₉₃) derived from FMuLV_{gag} (CCLCLTVFL) and the Fragment C-derived Th peptide p30 (FNNFTVSFWLRVPKVSASHLE) have been described previously.^{63,64} Peptide controls included the H-2 D^{b}-restricted HY peptide (WMHHNMDLI) derived from the Uty gene (Hy^{Db}Uty) and a tetanus toxin-derived H-2K^{b}-restricted peptide (SNWYFNHL) which is not encoded within these DNA vaccines.^{24,65} All peptides were synthesized commercially and supplied at >95% purity (Peptide Protein Research Ltd., Southampton, UK). Gag₈₅₋₉₃ peptide stocks (5 mM) were dissolved in DMSO, all other peptide stocks (1 mM) were dissolved in water; stocks were stored at −20° C.

Construction of DNA Vaccines

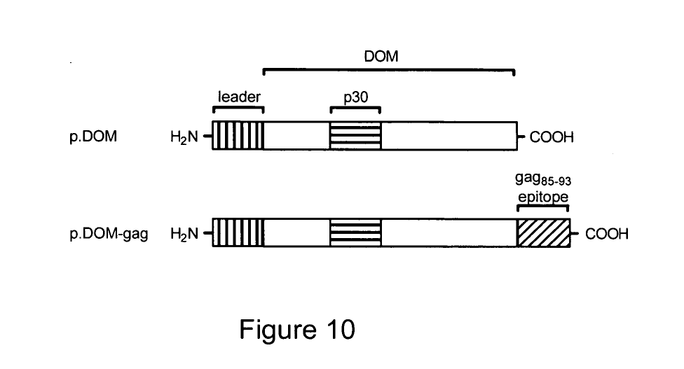

DNA vaccine design is indicated in FIG. 10. Construction of a DNA vaccine (p.DOM) containing the gene encoding the first domain (DOM) of FrC from tetanus toxin, with a leader sequence derived from the V_{H} of the IgM of the BCL₁ tumor, has been described previously.²⁴ The p.DOM vaccine was then used as a template to construct p.DOM-gag which encodes the first domain of FrC (DOM) with the sequence encoding the immunodominant H-2D^{b}-restricted FMuLV_{gag} CTL motif (gag₈₅₋₉₃) fused to the carboxyl terminus. Fusion to the C-terminus gives optimum processing and presentation. p.DOM-gag was constructed by PCR amplification of the first domain of FrC, encoded within p.DOM, using the forward primer 5′-TTTTAAGCTTGCCGCCACCATGGGTTGGAGC-3′ and the reverse primer 5′-TTTTGCGGCCGCTTACAGAAAAACAGTCAAACAGAGAC AACAGTTACCCCAGAAGTCACGCAGGAA-3′, which fuses the gag₈₅₋₉₃-encoding sequence to the 3′-terminus of DOM. The resulting PCR fragment was gel purified, digested and cloned into the expression vector pcDNA3 (Invitrogen Corp., San Diego, Calif.) using Hind III and Not I restriction sites. Both DNA constructs encode the BCL₁ leader sequence at the amino terminus; DNA vaccine stocks were prepared using the QIAfilter plasmid giga kit (Qiagen, Valencia, Calif.). Vaccine integrity was confirmed by DNA sequencing. Expression and product size were checked in vitro using the TNT® T7 Coupled Reticulocyte Lysate System (Promega Corp., Madison, Wis.).

Mice and Vaccination Protocol

The B6 gag-transgenic model, in which the gag protein from FMuLV is expressed under the control of the mouse albumin promoter in the liver, has been described previously.^{66,67,68} For DNA immunization, wild type B6 mice (wt) or gag transgenic mice (gag-Tg), bred in house, were vaccinated at 6-12 weeks of age with a total of 50 μg DNA in saline injected into two sites in the quadriceps muscles on day 0;⁷¹ mice were anesthetized and administered DNA vaccine booster injections together with electroporation at day 28, as described above. For cellular immunization, mice were injected with 1×10⁷ irradiated (10,000 rad) FBL-3 leukemia cells intraperitoneally on days 0 and 28. Animal experimentation was conducted within local Ethical Committee and UK Coordinating Committee for Cancer Research (UKCCCR, London, UK) guidelines, under Governmental (Home Office) license.

ELISpot

Following priming (day 0) and booster injections (day 28) splenocytes were harvested on day 36 and vaccine-specific IFNγ secretion by splenocytes from individual mice was assessed ex vivo (BD ELISpot Set, BD PharMingen, San Diego, Calif.), as described previously.⁴⁰ Splenocytes were incubated with either the H-2D^{b}-restricted gag₈₅₋₉₃ peptide for 24 h to assess CD8⁺ T cell responses or the p30 peptide (derived from the FrC fusion domain, DOM) was used to assess CD4⁺ T cell responses. Triplicate sample wells were tested with a range of gag₈₅₋₉₃ peptide concentrations; control samples were incubated without peptide. The reducing agent tris(2-carboxyethyl) phosphine hydrochloride (TCEP; Pierce Biotechnology, Rockford, Ill.), which has been shown to enhance the antigenicity of cysteine-containing synthetic peptides,⁷⁰ was included in each microtitre well (200 μM) during the 24 hour incubation stage. Peptide-specific ELISpot responses greater than 60 spot forming cells (SFC) per million splenocytes and more than twice baseline values observed in the absence of peptide were considered positive. To compare the frequency of T cells responding to different concentrations of the gag₈₅₋₉₃ peptide, as a measure of T cell avidity, baseline ELISpot responses without peptide were subtracted and the number of SFC/million splenocytes was calculated as a percentage of the maximum SFC/million splenocytes for each individual mouse. The data were then pooled within each experimental group to calculate the mean ELISpot response as a percentage of the maximum observed response for each peptide concentration.

Generation and Assay of Gag₈₅₋₉₃-Specific Cytotoxic CD8⁺ T Cells

To assess gag₈₅₋₉₃-specific CTL responses, vaccinated mice were sacrificed at day 36 and their spleens were removed. Single cell suspensions were made from individual spleens in complete medium. Splenocytes were washed, counted and resuspended at 3×10⁶ cells/ml: 15 ml were added to upright 25 cm² flasks together with recombinant human IL-2 (20 IU/ml, Perkin-Elmer, Foster City, Calif.), gag₈₅₋₉₃ peptide (0.01 μM) and 200 μM TCEP.⁷⁰ Following 6 days stimulation in vitro (37° C., 5% CO₂), cytolytic activity of the T cell cultures was assessed by standard 5 hour ⁵¹Cr-release assay as previously described,^{23,24} with target cells that were labeled with peptide/⁵¹Cr in the presence of 200 μM TCEP for 1 hour at 37° C. Targets included EL4 cells labeled with gag₈₅₋₉₃ peptide or control peptide (Uty), FBL-3 leukemia cells or NK-sensitive YAC-1 cells. Specific lysis was calculated by the standard formula of (release by CTL−spontaneous release)/(total release−spontaneous release)×100%). Spontaneous release was always <30%.

In Vivo Cytotoxicity Assay

Splenocytes were harvested from wt and gag-Tg mice (2×10⁷/ml in PBS) and cells from each strain were pulsed with 5 μM gag₈₅₋₉₃ peptide or control peptide (SNWYFNHL) for 30 minutes at 37° C. in the presence of 200 μM TCEP and washed in PBS. The gag and control peptide-pulsed cells were then incubated with 5 μM or 0.5 μM 5,6-carboxy-flourescein succinimidyl ester (CFSE) (Molecular Probes, Invitrogen Corp.), respectively, at room temperature for 8 minutes in the dark, and FCS (final concentration 20%) was added to quench the labeling reaction. After washing, syngeneic cells were mixed together, re-suspended in PBS and 2×10⁷ cells in 0.1 ml injected intravenously to each sex-matched, syngeneic recipient. Splenocytes were harvested from individual recipients after 20 hours and, following lysis of RBC, CFSE expression analyzed by FACSCalibur, using CELLQUEST software (BD Biosciences, San Diego, Calif.).

Tumor Challenge

Mice were challenged at day 36 following the first immunization by intraperitoneal injection of 5×10⁴ FBL-3 leukemia cells in PBS. All mice were monitored daily and were euthanized on detection of tumor development, in accordance with humane end point guidelines (UKCCCR).

Ex Vivo Intracellular IFNγ Assay

To assess priming of gag₈₅₋₉₃-specific CD8⁺ T cells mice were culled at day 36 following immunization and spleens harvested and processed for detection of intracellular IFNγ. Viable pooled splenocytes were selected by density centrifugation and B cells were removed using Mouse pan B Dynabeads® (Invitrogen Corp., Carlsbad, Calif.), according to the manufacturers instructions. Cells were incubated for 4 h at 37° C. in 96-well plates, at 1×10⁶ cells/well, in complete medium together with 200 μM TCEP, 10 U/well human recombinant IL-2, 1 μM gag₈₅₋₉₃ peptide or control peptide (SNWYFNHL) and 1 μl/well Golgi Plug (BD Biosciences). Following incubation samples were processed to label surface CD8 and intracellular IFNγ, as previously described,²³ prior to analysis by FACS.

Analysis of Autoimmune Injury

Following priming (day 0) and booster vaccinations (day 28) groups of B6 and gag-Tg mice were euthanized on day 36 to assess autoimmune injury. Control, naïve groups received no vaccinations. Liver samples were fixed in formaldehyde, paraffin-embedded, sectioned and stained with hematoxylin/eosin. Coded specimens were analyzed by a liver pathologist in a blinded manner for inflammation and lymphocyte infiltration using a Zeiss Axioskop 2 MOT microscope (Carl Zeiss Group, Oberkochen, Germany) and Zeiss Plan-NEOFLUAR 10×/0.30 objective lens. Images were recorded using a Zeiss AxioCam camera and Zeiss Axiovision 4 software with white balance correction provided by GIMP (GNU Image Manipulation Program) and processed with CorelDraw® Graphics Suite 12 (Corel Corporation, Ottawa, Canada).

Statistical Analysis

Experimental groups were compared using an unpaired, two-tailed t test. Survival curves were compared using the Chi square log-rank test. Experimental groups were considered significantly different from control groups if P<0.05.

DNA Vaccination Induces Gag₈₅₋₉₃-Specific CD8+ T Cells in Wt and Gag-Tg Mice.

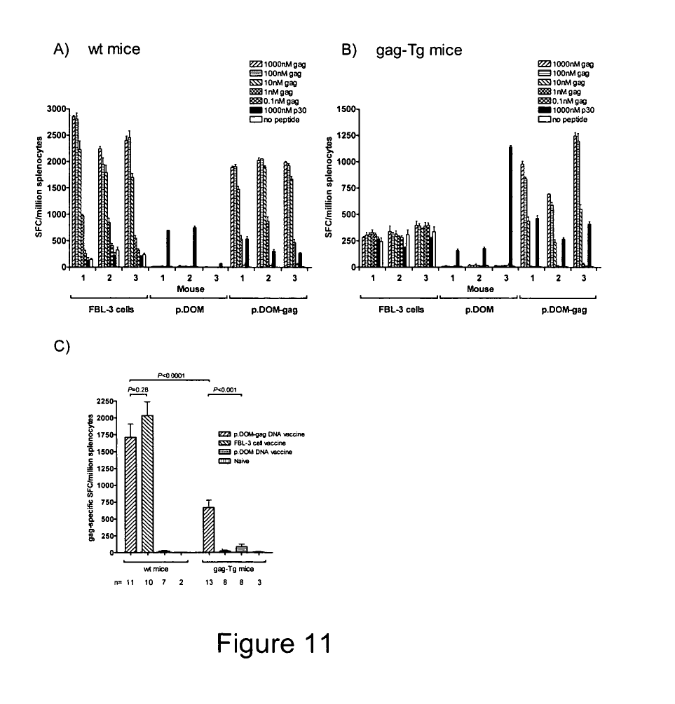

The ability of the p.DOM-gag DNA vaccine to induce CD8⁺ T cell responses to gag₈₅₋₉₃ was assessed by vaccinating wt and gag-Tg mice. For comparison, a control group was immunized with irradiated FBL-3 cells, which is known to induce a CD8⁺ T cell response specific for the immunodominant gag₈₅₋₉₃ epitope.^{71,63} T cell responses in the spleen were measured immediately ex vivo by ELISpot assay on day 36 (FIG. 11A). Vaccination with p.DOM-gag induced gag₈₅₋₉₃-specific T cell responses as measured by the production of IFNγ (FIG. 11A), with frequencies similar to those observed in FBL-3-vaccinated mice. The control DNA vaccine (p.DOM) generated no gag₈₅₋₉₃-specific responses (FIG. 11A). CD4⁺ Th cell responses against the ‘promiscuous’ MHC Class II-binding peptide p30, embedded in the FrC domain (DOM), were also detected in mice vaccinated with either DNA vaccine (p.DOM-gag or p.DOM), but not in mice immunized with irradiated FBL-3 cells, where the frequency of IFNγ-producing cells was equivalent to background levels (FIG. 11A).

Gag-Tg mice were also tested for their ability to respond to the p.DOM-gag DNA vaccine. This vaccine induced robust gag₈₅₋₉₃-specific T cell responses, as monitored by an ex vivo IFNγ ELISpot assay (FIG. 11B), although the frequency of responding cells was ˜2.5-fold lower than in wt mice (FIG. 11C). By contrast, immunization with irradiated FBL-3 tumor cells failed to induce detectable gag₈₅₋₉₃-specific T cell responses in gag-Tg mice (FIG. 11B), as we have previously reported.⁶⁶ A non-specific background response of cells producing IFNγ was observed in mice after immunization with FBL-3, but this did not reflect a response to the peptide and may be the consequence of an inflammatory response resulting from the injection of 1×10⁷ irradiated tumor cells 8 days prior to obtaining the spleen. The control p.DOM vaccine again induced no gag₈₅₋₉₃-specific responses, although CD4⁺ Th cell responses to the p30 peptide were detected in these animals, as well as those immunized with p.DOM-gag (FIG. 11B). Since the CD4⁺ T-cell response to p30 is derived from the unmanipulated mouse repertoire, this was used as an overall indicator of the DNA vaccine performance, which appeared effective in both wt and gag-Tg mice (Table I).

TABLE I
Ex vivo detection of vaccine-specific T lymphocytes in wt
and gag-Tg mice by tFNγ ELISpot assay.

Vaccine-specific responses:^{†}

| Vaccine | Peptide* | wt mice | gag-Tg mice |
|---|---|---|---|
| p.DOM-gag | gag₈₅₋₉₃ | 11/11 (100%) | 12/13 (92%) |
| | p30 | 10/11 (91%) | 13/13 (100%) |
| p.DOM | gag₈₅₋₉₃ | 0/7 (0%) | 0/8 (0%) |
| | p30 | 7/7 (100%) | 6/8 (75%) |
| Irradiated FBL-3 cells | gag₈₅₋₉₃ | 10/10 (100%) | 0/8 (0%) |
| | p30 | 0/10 (0%) | 0/8 (0%) |
| None | gag₈₅₋₉₃ | 0/2 (0%) | 0/3 (0%) |
| | p30 | 0/2 (0%) | 0/3 (0%) |

*Splenocytes incubated with 0.1 μM gag₈₅₋₉₃ peptide or 1 μM p30 peptide.
^{†}Data presented as: number of positive responders/total mice tested (% responding).

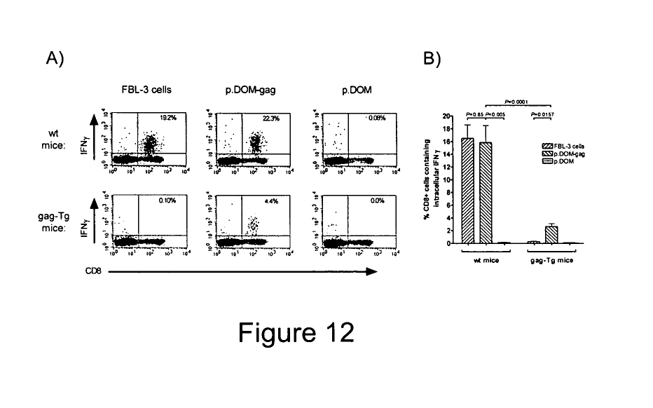

A survey of larger numbers of individual mice was then carried out, which demonstrated that although p.DOM-gag activated a lower frequency of gag₈₅₋₉₃-specific T cells in gag-Tg mice compared to wt mice (FIG. 11C), the number of animals responding was comparable between strains: 92% (12 of 13) of gag-Tg mice and 100% (11 of 11) of wt mice (Table 1). The efficacy of the vaccine was further assessed by FACS analysis to directly quantitate the number of gag₈₅₋₉₃-specific CD8⁺ T cells present in splenic lymphocytes, using anti-IFNγ (intracellular) and anti-CD8 (surface) antibodies (FIG. 12A). Although responses were again clearly evident in wt and gag-Tg mice, the mean percentage of splenic CD8⁺ T cells producing IFNγ was approximately six times lower in gag-Tg mice compared to wt mice (FIG. 12B).

Comparison of Avidity of Induced Gag₈₅₋₉₃-Specific CD8+ T Cells in Wt and Gag-Tg Mice

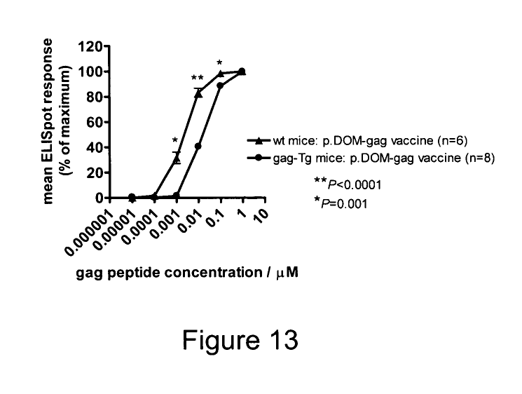

The frequency of gag₈₅₋₉₃-specific cells elicited in gag-Tg mice is low compared to wt mice probably due to central and peripheral tolerance mechanisms which could potentially have deleted high avidity gag₈₅₋₉₃-specific CD8+ T cells. To address this question we compared the frequency of CD8⁺ T cells from wt and gag-Tg mice that responded to varying concentrations of gag₈₅₋₉₃ peptide in an IFNγ ELISpot assay as a measure of T cell avidity. Wt and gag-Tg mice were vaccinated with either p.DOM-gag or the control DNA vaccine (p.DOM) and ELISpot responses were measured at day 36, as described above. Crucially, gag₈₅₋₉₃-specific CD8⁺ T cells elicited in the gag-Tg mice had ˜10-fold lower avidity compared to those from wt mice when tested against a range of gag₈₅₋₉₃ peptide concentrations ex vivo (FIG. 13). This difference, observed in two independent experiments comparing 11 wt and 12 gag-Tg mice, was reproducible and was highly statistically significant, with a p≦0.001.

Assessment of Autoimmunity in Vaccinated Gag-Tg Mice.

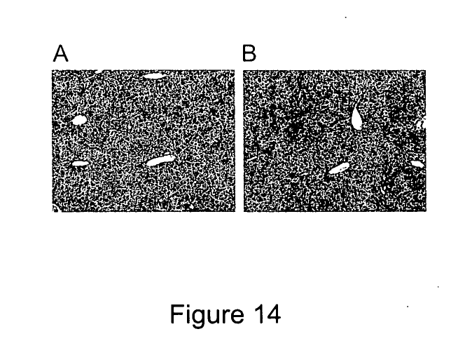

The presence of a population of activated gag₈₅₋₉₃-specific CD8⁺ T cells following DNA immunization could potentially result in autoimmune injury to hepatocytes expressing the FMuLV_{gag} protein. To assess this, blood was drawn from gag-Tg mice at day 36 following vaccination and serum levels of the liver enzymes AST and ALT measured as indicators of liver injury. In addition, mice were sacrificed at this time point for blinded histological analysis of liver tissue. All animals appeared healthy with no evidence of increased ALT/AST serum levels or autoimmune hepatocyte injury by histologic analysis of liver sections in vaccinated gag-Tg animals (FIG. 14).

Cytotoxic Activity In Vitro of Gag₈₅₋₉₃-Specific CD8⁺ T Cells Induced in Wt and Gag-Tg Mice.

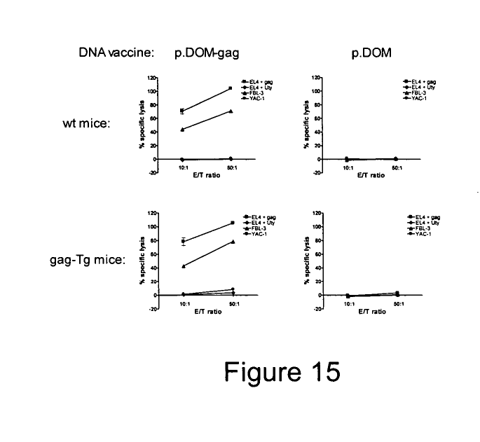

The absence of autoimmune injury in vaccinated gag-Tg mice could reflect resistance of the liver to CD8⁺ T cell effector activity or the induction of a not fully competent response in these hosts. Therefore, the ability of the CD8⁺ T cell response induced by the p.DOM-gag DNA vaccine in gag-Tg mice to exhibit lytic activity against targets expressing the gag₈₅₋₉₃ epitope was tested. At day 36 after vaccination, splenocytes from wt or gag-Tg mice were stimulated in vitro with peptide for 6 days and lytic activity assessed in a ⁵¹Cr release assay. CTL from either wt or gag-Tg mice, primed with p.DOM-gag, lysed EL4 target cells pulsed with gag₈₅₋₉₃ peptide but not an irrelevant H-2D^{b}-restricted peptide (FIG. 15). CTL from either strain also lysed FBL-3 leukemia cells expressing endogenous FMuLV_{gag} antigen (FIG. 15). No specific CTL activity was generated by culture of splenocytes in vitro with gag₈₅₋₉₃ peptide following vaccination with the control vaccine, p.DOM, and no lytic activity was observed against the NK-susceptible cell line YAC-1 (FIG. 15).

Cytotoxic Activity In Vivo of Gag₈₅₋₉₃-Specific CD8+ T Cells Induced in Wt and Gag-Tg Mice.

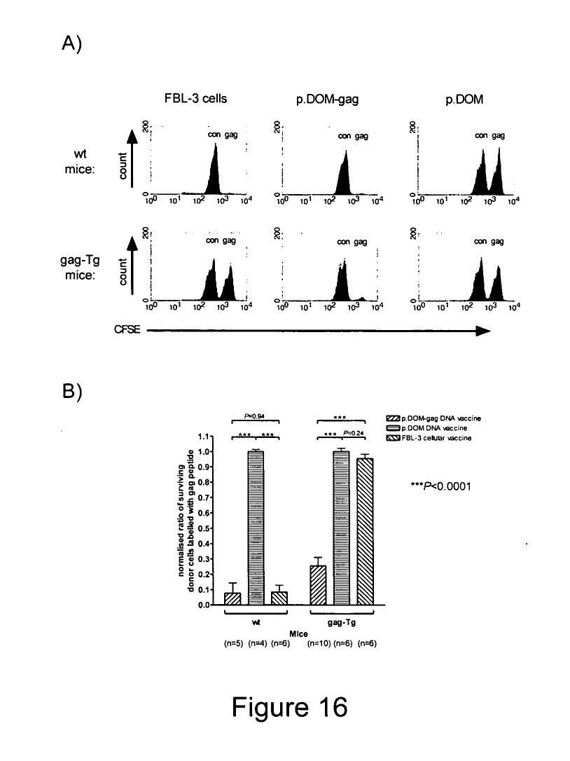

The above studies demonstrated that gag₈₅₋₉₃-specific lytic activity could be elicited following in vitro stimulation of the CD8⁺ T cells that had been induced in gag-Tg mice, but the absence of autoimmune injury suggested that the cells might not be expressing such lytic activity in vivo in the absence of re-stimulation under in vitro conditions. To address this, wt and gag-Tg mice were vaccinated with either p.DOM-gag, the control DNA vaccine (p.DOM), or irradiated FBL-3 cells. At day 36, mice were injected intravenously with sex-matched, syngeneic splenocyte targets pulsed with either gag₈₅₋₉₃ peptide or control peptide that had been differentially labeled with CFSE to permit distinction between the two targets by flow cytometry. Wt mice that had previously been vaccinated with either p.DOM-gag or irradiated FBL-3 cells rapidly and efficiently lysed target splenocytes pulsed with the gag₈₅₋₉₃ peptide, as reflected by the clearance of >90% of the CFSE^{hi} targets within 20 hours, but not those from the co-transferred population pulsed with the control peptide (CFSE^{low}) (FIG. 16A). Both populations of CFSE-labeled target cells survived in wt mice given the control DNA vaccine, p.DOM (FIG. 16A).

Notably, in gag-Tg mice vaccinated with p.DOM-gag, despite the absence of ongoing liver toxicity, the gag₈₅₋₉₃ peptide-pulsed target cells were similarly eliminated in a peptide-specific manner (FIG. 16A). The degree of specific target cell lysis did not differ significantly between wt and gag-Tg strains immunized with this DNA vaccine (FIG. 16B, P=0.073), indicating that, for target cells pulsed with high concentrations of the peptide epitope, the reduced avidity of the CTL elicited in the transgenic mice did not markedly affect their performance. In contrast to wt mice, gag-Tg mice vaccinated with irradiated FBL-3 cells failed to lyse target cells labeled with the gag₈₅₋₉₃ peptide (FIGS. 16A, B), confirming the inability of this approach to induce gag₈₅₋₉₃-specific CTL in these animals. Both populations of CFSE-labeled target cells survived in gag-Tg mice given the control DNA vaccine, p.DOM (FIGS. 16A, B).

Induction of Gag₈₅₋₉₃-Specific CTL by p.DOM-Gag Protects Against FBL-3 Leukemia Growth In Vivo in Wt and Gag-Tg Mice.

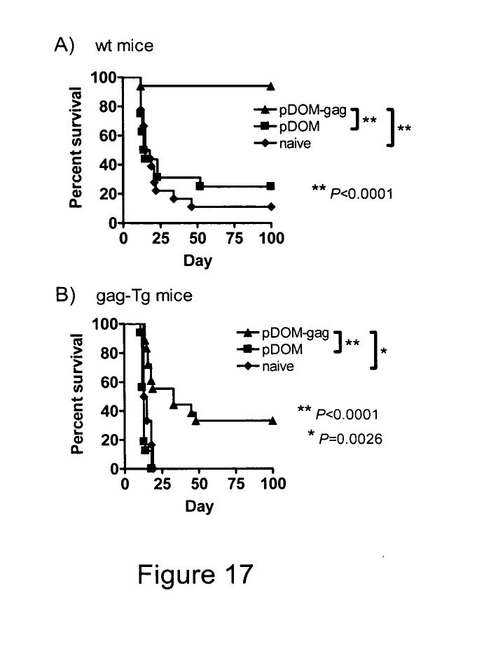

Although the induced gag₈₅₋₉₃-specific CTL demonstrated the ability to lyse peptide-pulsed targets in vivo, the lower avidity of this CD8+ T cell response induced in gag-Tg mice as compared to wt mice might make this response inadequate to recognize and protect the mice from leukemia in vivo. This represents the typical challenge that might be anticipated for targeting human tumor-associated antigens, in which the candidate antigen is detected in normal tissues but over-expressed in the malignancy. To address this, wt or gag-Tg mice were vaccinated with p.DOM-gag or the control vaccine (p.DOM) and then challenged at day 36 by intraperitoneal injection of 5×10⁴ FBL-3 leukemia cells. Immunization with p.DOM-gag afforded significant protection from leukemia in wt mice, with ˜95% surviving, compared to naïve animals or those given the control vaccine (FIG. 17A). Interestingly, a low level of protection (≦25%) was occasionally observed in control wt groups (naïve mice or those given the control vaccine, p.DOM), suggesting that injection of this dose of FBL-3 leukemia cells alone can lead to the spontaneous induction of natural protective immunity in wt mice (FIG. 17A).

Vaccination with p.DOM-gag also led to significant protection in gag-Tg mice (FIG. 17B). In these mice, there was no evidence for spontaneous induction of immunity following injection of FBL-3 cells, since all naive or control vaccinated mice succumbed by day 19 (FIG. 17B). Protection by the p.DOM-gag vaccine therefore demonstrates the ability of the lower avidity gag₈₅₋₉₃-specific CTL to destroy FBL-3 leukemia cells in vivo, with ˜⅓ of the gag-Tg mice vaccinated with p.DOM-gag exhibiting prolongation of survival (FIG. 17B).

Summary

The majority of known human tumor-associated antigens derive from non-mutated self-proteins. T-cell tolerance, essential to prevent autoimmunity, must therefore be cautiously circumvented to generate cytotoxic T-cell responses against these targets. This example uses DNA fusion vaccines to activate high levels of peptide-specific CTL. Key foreign sequences from tetanus toxin activate tolerance-breaking CD4⁺ T-cell help. Candidate MHC Class I-binding tumor peptide sequences are fused to the C-terminus for optimal processing and presentation.

The provision of heterologous T-cell help within the vaccine is aimed to circumvent tolerance to tumor antigens in the CD4+ T-cell arm. CD8⁺ T cells which receive help at priming are better equipped to expand and to resist apoptosis on second encounter with antigen, thereby improving the quality and longevity of the CTL response.^{72,73,74} Although tumor-specific T-cell help might be required for maintenance of the CD8⁺ T cells,⁷⁵ our experience has been that challenge with tumor cells can expand tumor-specific CD8⁺ T cells that have previously been primed by DNA vaccination.²³ However, we are investigating the effect on CD8+ T cell priming of encoding both tumor-derived CD4+ and CD8+ T-cell epitopes within our DNA vaccines.

In addition to providing heterologous T cell help, our DNA fusion vaccine encodes a CTL epitope derived from the target leukemia. The single epitope design allows a focused CTL response, reducing the risk of cross-reactive autoimmunity. However, targeting several epitopes derived from the same or an alternative antigen would be advantageous and would reduce the likelihood of tumor escape due to antigenic mutation or deletion. To avoid immunodominance effects, delivery of the second vaccine could be into a separate site. An integrated attack on multiple epitopes expressed by leukemic cells could compensate for the loss of antigen-specific T cell frequency and avidity observed in this tolerized repertoire and improve survival.

To model performance against a leukemia-associated antigen in a tolerized setting, we constructed a fusion vaccine encoding an immunodominant CTL epitope derived from Friend Murine Leukemia Virus gag protein (FMuLV_{gag}) and vaccinated tolerant FMuLV_{gag}-transgenic mice. Vaccination induced epitope-specific IFNγ-producing CD8⁺ T cells in normal and FMuLV_{gag}-transgenic mice; the frequency and avidity of activated cells were reduced in the latter, with no evidence of autoimmune injury. However, effector CD8⁺ T cells activated from either repertoire acquired peptide-specific cytotoxicity in vitro and in vivo. CTL were able to kill FBL-3 leukemia cells expressing endogenous FMuLV_{gag} antigen in vitro, and to protect against leukemia challenge in vivo. These results demonstrate a simple strategy to engage anti-microbial T-cell help to activate polyclonal lower avidity but still leukemia-reactive CTL from a tolerized repertoire.

### REFERENCES

1. Sedegah, M., R. Hedstrom, P. Hobart, and S. L. Hoffman. 1994. Protection against malaria by immunization with plasmid DNA encoding circumsporozoite protein. Proc. Natl. Acad. Sci. USA 91: 9866-9870.
2. Polack, F. P., S. H. Lee, S. Permar, E. Manyara, H. G. Nousari, Y. Jeng, F. Mustafa, A. Valsamakis, R. J. Adams, H. L. Robinson, and D. E. Griffin. 2000. Successful DNA immunization against measles: neutralizing antibody against either the hemagglutinin or fusion glycoprotein protects rhesus macaques without evidence of atypical measles. Nat. Med. 6: 776-781.
3. Ulmer, J. B., J. J. Donnelly, S. E. Parker, G. H. Rhodes, P. L. Feigner, V. J. Dwarki, S. H. Gromkowski, R. R. Deck, C. M. DeWitt, A. Friedman, et al. 1993. Heterologous protection against influenza by injection of DNA encoding a viral protein. Science 259: 1745-1749.
4. Davis, H. L., M. L. Michel, and R. G. Whalen. 1993. DNA-based immunization induces continuous secretion of hepatitis B surface antigen and high levels of circulating antibody. Hum. Mol. Genet. 2: 1847-1851.
5. Conry, R. M., A. F. LoBuglio, J. Kantor, J. Schlom, F. Loechel, S. E. Moore, L. A. Sumerel, D. L. Barlow, S. Abrams, and D. T. Curiel. 1994. Immune response to a carcinoembryonic antigen polynucleotide vaccine. Cancer Res. 54: 1164-1168.
6. Hawkins, R. E., D. Zhu, M. Ovecka, G. Winter, T. J. Hamblin, A. Long, and F. K. Stevenson. 1994. Idiotypic vaccination against human B-cell lymphoma: rescue of variable region gene sequences from biopsy material for assembly as single-chain Fv personal vaccines. Blood 83: 3279-3288.
7. Ciernik, I. F., J. A. Berzofsky, and D. P. Carbone. 1996. Induction of cytotoxic T lymphocytes and antitumor immunity with DNA vaccines expressing single T cell epitopes. J. Immunol. 156: 2369-2375.
8. Spellerberg, M. B., D. Zhu, A. Thompsett, C. A. King, T. J. Hamblin, and F. K. Stevenson. 1997. DNA vaccines against lymphoma: promotion of antiidiotypic antibody responses induced by single chain Fv genes by fusion to tetanus toxin fragment C. J. Immunol. 159: 1885-1892.
9. Hsu, C. H., K. Y. Chua, M. H. Tao, Y. L. Lai, H. D. Wu, S. K. Huang, and K. H. Hsieh. 1996. Immunoprophylaxis of allergen-induced immunoglobulin E synthesis and airway hyperresponsiveness in vivo by genetic immunization. Nat. Med. 2: 540-544.
10. Wildbaum, G., M. A. Nahir, and N. Karin. 2003. Beneficial autoimmunity to proinflammatory mediators restrains the consequences of self-destructive immunity. Immunity 19: 679-688.
11. Youssef, S., G. Maor, G. Wildbaum, N. Grabie, A. Gour-Lavie, and N. Karin. 2000. C—C chemokine-encoding DNA vaccines enhance breakdown of tolerance to their gene products and treat ongoing adjuvant arthritis. J. Clin. Invest. 106: 361-371.
12. Gurunathan, S., D. M. Klinman, and R. A. Seder. 2000. DNA vaccines: immunology, application, and optimization. Annu. Rev. Immunol. 18: 927-974.
13. King, C. A., M. B. Spellerberg, D. Zhu, J. Rice, S. S. Sahota, A. R. Thompsett, T. J. Hamblin, J. Radl, and F. K. Stevenson. 1998. DNA vaccines with singlechain Fv fused to fragment C of tetanus toxin induce protective immunity against lymphoma and myeloma. Nat. Med. 4: 1281-1286.
14. Liu, M. A., W. McClements, J. B. Ulmer, J. Shiver, and J. Donnelly. 1997. Immunization of non-human primates with DNA vaccines. Vaccine 15: 909-912.
15. Turnes, C. G., J. A. Aleixo, A. V. Monteiro, and O. A. Dellagostin. 1999. DNA inoculation with a plasmid vector carrying the faeG adhesin gene of Escherichia coli K88ab induced immune responses in mice and pigs. Vaccine 17: 2089-2095.
16. Jones, T. R., D. L. Narum, A. S. Gozalo, J. Aguiar, S. R. Fuhrmann, H. Liang, J. D. Haynes, J. K. Moch, C. Lucas, T. Luu, et al. 2001. Protection of Aotus monkeys by Plasmodium falciparum EBA-175 region 11 DNA prime-protein boost immunization regimen. J. Infect. Dis. 183: 303-312.
17. Robinson, H. L., D. C. Montefiori, R. P. Johnson, K. H. Manson, M. L. Kalish, J. D. Lifson, T. A. Rizvi, S. Lu, S. L. Hu, G. P. Mazzara, et al. 1999. Neutralizing antibody-independent containment of immunodeficiency virus challenges by DNA priming and recombinant pox virus booster immunizations. Nat. Med. 5: 526-534.
18. Li, S., M. Rodrigues, D. Rodriguez, J. R. Rodriguez, M. Esteban, P. Palese, R. S. Nussenzweig, and F. Zavala. 1993. Priming with recombinant influenza virus followed by administration of recombinant vaccinia virus induces CD8⁺ T-cell-mediated protective immunity against malaria. Proc. Natl. Acad. Sci. USA 90: 5214-5218.
19. McConkey, S. J., W. H. Reece, V. S. Moorthy, D. Webster, S. Dunachie, G. Butcher, J. M. Vuola, T. J. Blanchard, P. Gothard, K. Watkins, et al. 2003. Enhanced T-cell immunogenicity of plasmid DNA vaccines boosted by recombinant modified vaccinia virus Ankara in humans. Nat. Med. 9: 729-735.
20. Stevenson, F. K., J. Rice, and D. Zhu. 2004. Tumor vaccines. Adv. Immunol. 82: 49-103.
21. Stevenson, F. K., J. Rice, C. H. Ottensmeier, S. M. Thirdborough, and D. Zhu. 2004. DNA fusion gene vaccines against cancer: from the laboratory to the clinic. Immunol. Rev. 199: 156-180.
22. Thirdborough, S. M., J. N. Radcliffe, P. S. Friedmann, and F. K. Stevenson. 2002. Vaccination with DNA encoding a single-chain TCR fusion protein induces anticlonotypic immunity and protects against T-cell lymphoma. Cancer Res. 62: 1757-1760.
23. Rice, J., S. Buchan, and F. K. Stevenson. 2002. Critical components of a DNA fusion vaccine able to induce protective cytotoxic T cells against a single epitope of a tumor antigen. J. Immunol. 169: 3908-3913.
24. Rice, J., T. Elliott, S. Buchan, and F. K. Stevenson. 2001. DNA fusion vaccine designed to induce cytotoxic T cell responses against defined peptide motifs: implications for cancer vaccines. J. Immunol. 167: 1558-1565.
25. Kadowaki, S., Z. Chen, H. Asanuma, C. Aizawa, T. Kurata, and S. Tamura. 2000. Protection against influenza virus infection in mice immunized by administration of hemagglutinin-expressing DNAs with electroporation. Vaccine 18: 2779-2788.
26. Dupuis, M., K. Denis-Mize, C. Woo, C. Goldbeck, M. J. Selby, M. Chen, G. R. Otten, J. B. Ulmer, J. J. Donnelly, G. Ott, and D. M. McDonald. 2000. Distribution of DNA vaccines determines their immunogenicity after intramuscular injection in mice. J. Immunol. 165: 2850-2858.
27. Widera, G., M. Austin, D. Rabussay, C. Goldbeck, S. W. Barnett, M. Chen, L. Leung, G. R. Otten, K. Thudium, M. J. Selby, and J. B. Ulmer. 2000. Increased DNA vaccine delivery and immunogenicity by electroporation in vivo. J. Immunol. 164: 4635-4640.
28. Babiuk, L. A., R. Pontarollo, S. Babiuk, B. Loehr, and S. van Drunen Littel-van den Hurk. 2003. Induction of immune responses by DNA vaccines in large animals. Vaccine 21: 649-658.
29. Aihara, H., and J. Miyazaki. 1998. Gene transfer into muscle by electroporation in vivo. Nat. Biotechnol. 16: 867-870.
30. Mir, L. M., M. F. Bureau, J. Gehl, R. Rangara, D. Rouy, J. M. Caillaud, P. Delaere, D. Branellec, B. Schwartz, and D. Scherman. 1999. High-efficiency gene transfer into skeletal muscle mediated by electric pulses. Proc. Natl. Acad. Sci. USA 96: 4262-4267.
31. Mathiesen, I. 1999. Electropermeabilization of skeletal muscle enhances gene transfer in vivo. Gene Ther. 6: 508-514.
32. Babiuk, S., M. E. Baca-Estrada, M. Foldvari, M. Storms, D. Rabussay, G. Widera, and L. A. Babiuk. 2002.
   Electroporation improves the efficacy of DNA vaccines in large animals. Vaccine 20: 3399-3408.
33. Tollefsen, S., T. Tjelle, J. Schneider, M. Harboe, H. Wiker, G. Hewinson, K. Huygen, and Mathiesen. 2002. Improved cellular and humoral immune responses against Mycobacterium tuberculosis antigens after intramuscular DNA immunization combined with muscle electroporation. Vaccine 20: 3370-3378.
34. George, A. J., A. L. Tutt, and F. K. Stevenson. 1987. Anti-idiotypic mechanisms involved in suppression of a mouse B cell lymphoma, BCL1. J. Immunol. 138: 628-634.
35. Huang, A. Y., P. H. Gulden, A. S. Woods, M. C. Thomas, C. D. Tong, W. Wang, V. H. Engelhard, G. Pasternack, R. Cotter, D. Hunt, et al. 1996. The immunodominant major histocompatibility complex class I-restricted antigen of a murine colon tumor derives from an endogenous retroviral gene product. Proc. Natl. Acad. Sci. USA 93: 9730-9735.
36. Illidge, T., J. Honeychurch, W. Howatt, F. Ross, B. Wilkins, and M. Cragg. 2000. A new in vivo and in vitro B cell lymphoma model, pi-BCL1. Cancer Biother. Radiopharm. 15: 571-580.
37. Benvenuti, F. O. R. Burrone, and D. G. Efremov. 2000. Anti-idiotypic DNA vaccines for lymphoma immunotherapy require the presence of both variable region genes for tumor protection. Gene Ther. 7: 605-611.
38. George, A., S. Folkard, T. Hamblin, and F. Stevenson. 1988. Idiotypic vaccination as a treatment for a B cell lymphoma. J. Immunol. 141: 2168-2174.
39. Campbell, M. J., W. Carroll, S. Kon, K. Thielemans, J. B. Rothbard, S. Levy, and R. Levy. 1987. Idiotype vaccination against murine B cell lymphoma: humoral and cellular responses elicited by tumor-derived immunoglobulin M and its molecular subunits. J. Immunol. 139: 2825-2833.
40. Rice, J., S. Buchan, H. Dewchand, E. Simpson, and F. K. Stevenson. 2004. DNA fusion vaccines induce targeted epitope-specific cytotoxic T lymphocytes against minor histocompatibility antigens from a normal or tolerized repertoire. J. Immunol. 173: 4492-4497.
41. MacGregor, R. R., J. D. Boyer, K. E. Ugen, K. E. Lacy, S. J. Gluckman, M. L. Bagarazzi, M. A. Chattergoon, Y. Baine, T. J. Higgins, R. B. Ciccarelli, et al. 1998. First human trial of a DNA-based vaccine for treatment of human immunodeficiency virus type I infection: safety and host response. J. Infect. Dis. 178: 92-100.
42. Wang, R., D. L. Doolan, T. P. Le, R. C. Hedstrom, K. M. Coonan, Y. Charoenvit, T. R. Jones, P. Hobart, M. Margalith, J. Ng, et al. 1998. Induction of antigen-specific cytotoxic T lymphocytes in humans by a malaria DNA vaccine. Science 282: 476-480.
43. Wang, R., J. Epstein, F. M. Baraceros, E. J. Gorak, Y. Charoenvit, D. J. Carucci, R. C. Hedstrom, N. Rahardjo, T. Gay, P. Hobart, et al. 2001. Induction of CD4⁺ T cell-dependent CD8⁺ type I responses in humans by a malaria DNA vaccine. Proc. Natl. Acad. Sci. USA 98: 10817-10822.
44. Gronevik, E., S. Tollefsen, L. I. Sikkeland, T. Haug, T. E. Tjelle, and I. Mathiesen. 2003. DNA transfection of mononuclear cells in muscle tissue. J. Gene Med. 5: 909-917.
45. Uno-Furuta, S., S. Tamaki, Y. Takebe, S. Takamura, A. Kamei, G. Kim, I. Kuromatsu, M. Kaito, Y. Adachi, and Y. Yasutomi. 2001. Induction of virus specific cytotoxic T lymphocytes by in vivo electric administration of peptides. Vaccine 19: 2190-2196.
46. Dayball, K., J. Millar, M. Miller, Y. H. Wan, and J. Bramson. 2003. Electroporation enables plasmid vaccines to elicit CD8⁺ T cell responses in the absence of CD4⁺ T cells. J. Immunol. 171: 3379-3384.
47. Zinkernagel, R. M., S. Ehl, P. Aichele, S. Oehen, T. Kundig, and H. Hengartner. 1997. Antigen localization regulates immune responses in a dose- and time-dependent fashion: a geographical view of immune reactivity. Immunol. Rev. 156: 199-209.
48. Farr, R. S., and F. J. Dixon, Jr. 1960. The effect of antigen concentration on the initiation of detectable antibody synthesis in rabbits. J. Immunol. 85: 250-257.
49. Schneider, J., S. C. Gilbert, C. M. Hannan, P. Degano, E. Prieur, E. G. Sheu, M. Plebanski, and A. V. Hill. 1999. Induction of CD8+ T cells using heterologous prime-boost immunization strategies. Immunol. Rev. 170: 29-38.
50. Zavala, F., M. Rodrigues, D. Rodriguez, J. R. Rodriguez, R. S. Nussenzweig, and M. Esteban. 2001. A striking property of recombinant poxviruses: efficient inducers of in vivo expansion of primed CD8⁺ T cells. Virology 280: 155-159.
51. Blanchard, T. J., A. Alcami, P. Andrea, and G. L. Smith. 1998. Modified vaccinia virus Ankara undergoes limited replication in human cells and lacks several immunomodulatory proteins: implications for use as a human vaccine. J. Gen. Virol. 79: 1159-1167.
52. Cooney, E. L., A. C. Collier, P. D. Greenberg, R. W. Coombs, J. Zarling, D. E. Arditti, M. C. Hoffman, S. L. Hu, and L. Corey. 1991. Safety of and immunological response to a recombinant vaccinia virus vaccine expressing HIV envelope glycoprotein. Lancet 337: 567-572.
53. Kundig, T. M., C. P. Kalberer, H. Hengartner, and R. M. Zinkernagel. 1993. Vaccination with two different vaccinia recombinant viruses: long-term inhibition of secondary vaccination. Vaccine 11:1154-1158.
54. Rooney, J. F., C. Wohlenberg, K. J. Cremer, B. Moss, and A. L. Notkins. 1988. Immunization with a vaccinia virus recombinant expressing herpes simplex virus type I glycoprotein D: long-term protection and effect of revaccination. J. Virol. 62: 1530-1534.
55. Yewdell, J. W., and J. R. Bennink. 1999. Immunodominance in major histocompatibility complex class I-restricted T lymphocyte responses. Annu. Rev. Immunol. 17: 51-88.
56. Harrington, L. E., R. Most R V, J. L. Whitton, and R. Ahmed. 2002. Recombinant vaccinia virus-induced T-cell immunity: quantitation of the response to the virus vector and the foreign epitope. J. Virol. 76: 3329-3337.
57. Palmowski, M. J., E. M. Choi, I. F. Hermans, S. C. Gilbert, J. L. Chen, U. Gileadi, M. Salio, A. Van Pel, S. Man, E. Bonin, et al. 2002. Competition between CTL narrows the immune response induced by prime-boost vaccination protocols. J. Immunol. 168: 4391-4398.
58. Anderson, R. J., C. M. Hannan, S. C. Gilbert, S. M. Laidlaw, E. G. Sheu, S. Korten, R. Sinden, G. A. Butcher, M. A. Skinner, and A. V. Hill. 2004. Enhanced CD8⁺ T cell immune responses and protection elicited against Plasmodium berghei malaria by prime boost immunization regimens using a novel attenuated fowlpox virus. J. Immunol. 172: 3094-3100.
59. Chastain, M., A. J. Simon, K. A. Soper, D. J. Holder, D. L. Montgomery, S. L. Sagar, D. R. Casimiro, and C. R. Middaugh. 2001. Antigen levels and antibody titers after DNA vaccination. J. Pharm. Sci. 90: 474-484.
60. Mir, L. M. 2001. Therapeutic perspectives of in vivo cell electropermeabilization. Bioelectrochemistry 53: 1-10.
61. Kjeken, R., T. E. Tjelle, D. Kvale, and I. Mathiesen. 2004. Clinical evaluation of pain and muscle damage induced by electroporation of skeletal muscle in humans. In 7th Annual Meeting of the American Society of Gene Therapy, Jun. 2-6, 2004.
62. Greenberg P D, Kern D E, Cheever M A. Therapy of disseminated murine leukemia with cyclophosphamide and immune Lyt-1+2-T cells. Tumor eradication does not require participation of cytotoxic T cells. J Exp Med. 1985; 161:1122-1134
63. Chen W, Qin H, Chesebro B, Cheever M A. Identification of a gag-encoded cytotoxic T-lymphocyte epitope from FBL-3 leukemia shared by Friend, Moloney, and Rauscher murine leukemia virus-induced tumors. J. Virol. 1996; 70:7773-7782
64. Panina-Bordignon P, Tan A, Termijtelen A, Demotz S, Corradin G, Lanzavecchia A. Universally immunogenic T cell epitopes: promiscuous binding to human MHC class II and promiscuous recognition by T cells. Eur J. Immunol. 1989; 19:2237-2242
65. Greenfield A, Scott D, Pennisi D, Ehrmann I, Ellis P, Cooper L, Simpson E, Koopman P. An H-YDb epitope is encoded by a novel mouse Y chromosome gene. Nat Genet. 1996; 14:474-478
66. Ohlen C, Kalos M, Hong D J, Shur A C, Greenberg P D. Expression of a tolerizing tumor antigen in peripheral tissue does not preclude recovery of high-affinity CD8+ T cells or CTL immunotherapy of tumors expressing the antigen. J. Immunol. 2001; 166:2863-2870
67. Teague R M, Sather B D, Sacks J A, Huang M Z, Dossett M L, Morimoto J, Tan X, Sutton S E, Cooke M P, Ohlen C, Greenberg P D. Interleukin-15 rescues tolerant CD8+ T cells for use in adoptive immunotherapy of established tumors. Nat Med. 2006; 12:335-341
68. Ohlen C, Kalos M, Cheng L E, Shur A C, Hong D J, Carson B D, Kokot N C, Lerner C G, Sather B D, Huseby E S, Greenberg P D. CD8(+) T cell tolerance to a tumor-associated antigen is maintained at the level of expansion rather than effector function. J Exp Med. 2002; 195:1407-1418
69. Firat H, Garcia-Pons F, Tourdot S, Pascolo S, Scardino A, Garcia Z, Michel M L, Jack R W, Jung G, Kosmatopoulos K, Mateo L, Suhrbier A, Lemonnier F A, Langlade-Demoyen P. H-2 class I knockout, HLA-A2.1-transgenic mice: a versatile animal model for preclinical evaluation of antitumor immunotherapeutic strategies. Eur J. Immunol 1999; 29:3112-3121
70. Chen W, Yewdell J W, Levine R L, Bennink J R. Modification of cysteine residues in vitro and in vivo affects the immunogenicity and antigenicity of major histocompatibility complex class I-restricted viral determinants. J Exp Med. 1999; 189:1757-1764
71. Klarnet J P, Kern D E, Okuno K, Holt C, Lilly F, Greenberg P D. FBL-reactive CD8+ cytotoxic and CD4+ helper T lymphocytes recognize distinct Friend murine leukemia virus-encoded antigens. J Exp Med. 1989; 169:457-467
72. Shedlock D J, Shen H. Requirement for CD4 T cell help in generating functional CD8 T cell memory. Science. 2003; 300:337-339
73. Sun J C, Bevan M J. Defective CD8 T cell memory following acute infection without CD4 T cell help. Science. 2003; 300:339-342
74. Janssen E M, Lemmens E E, Wolfe T, Christen U, von Herrath M G, Schoenberger S P. CD4+ T cells are required for secondary expansion and memory in CD8+ T lymphocytes. Nature. 2003; 421:852-856
75. Ossendorp F, Mengede E, Camps M, Filius R, Melief C J. Specific T helper cell requirement for optimal induction of cytotoxic T lymphocytes against major histocompatibility complex class II negative tumors. J Exp Med. 1998; 187:693-702

Although the present invention has been described in detail with reference to examples above, it is understood that various modifications can be made without departing from the spirit of the invention. Accordingly, the invention is limited only by the following claims. All cited patents, applications and publications referred to in this application are herein incorporated by reference in their entirety.

1. A method of boosting an immune response in an individual to an antigen, said individual having been previously primed against or exposed to said antigen, said method comprising administering to said individual a nucleic acid construct encoding said antigen by electroporation.

2. A method according to claim 1 wherein the individual has been previously primed against said antigen as a result of administration of said antigen.

3. A method according to claim 2 wherein the antigen was previously administered to said individual as a nucleic acid construct encoding said antigen.

4. A method according to claim 2 wherein said antigen was previously administered to said individual as a polypeptide comprising said antigen.

5. A method according to claim 1 wherein the nucleic acid construct is selected from the group consisting of DNA, RNA and cDNA.

6. A method according to claim 1 wherein the nucleic acid construct encodes said antigen and a further immunomodulatory polypeptide.

7. A method according to claim 6 wherein the further immunomodulatory polypeptide acts as an adjuvant.

8. A method according to claim 7 wherein the further immunomodulatory polypeptide is an immunogenic pathogen derived sequence.

9. A method according to claim 8 wherein the immunogenic pathogen derived sequence is selected from the group consisting of tetanus toxoid fragment C (Frc) or a component thereof, plant viral coat protein and cytokines.

10. A method according to claim 1 wherein antigen is derived from a pathogen.

11. A method according to claim 10 wherein the pathogen is selected from the group consisting of a virus and a bacteria.

12. A method according to claim 1 wherein the antigen is derived from a self or altered self-polypeptide.

13. A method according to claim 12 wherein the self or altered self-polypeptide is associated with an autoimmune disease.

14. A method according to claim 12 wherein the self or altered self-polypeptide is a tumor antigen.

15. A method according to claim 14 wherein the antigen is selected from the group consisting of an antigen derived from colon carcinoma, an antigen derived from a B-cell lymphoma and an antigen derived from leukaemia.

16. A method according to claim 1 wherein the antigen is capable of inducing an immune response selected from the group consisting of a CD4+ T-cell response, a CD8+ T-cell response, a cytotoxic T lymphocyte (CTL) response and an antibody response.

17. A method of inducing an immune response to an antigen in an individual, said method comprising the steps of
firstly administering to said individual a priming antigen by a non-electroporation method; and
secondly administering to said individual a boosting antigen by electroporation;
wherein the boosting antigen is administered in the form of a nucleic acid construct capable of encoding the antigen in vivo.

18. A method according to claim 17 wherein the priming antigen is in the form of a nucleic acid construct encoding said antigen.

19. A method according to claim 17 wherein the priming antigen is a polypeptide comprising said antigen.

20. A method according to claim 17 wherein the priming antigen is administered by intramuscular injection.

21. A method according to claim 17 wherein the antigen is capable of inducing an immune response selected from the group consisting of a CD4+ T-cell response, a CD8+ T-cell response, a cytotoxic T lymphocyte (CTL) response and an antibody response.

22. A method according to claim 17 wherein the nucleic acid construct is selected from the group consisting of DNA, RNA and cDNA.

23. A method according to claim 17 wherein the nucleic acid construct encodes said antigen and a further immunomodulatory polypeptide.

24. A method according to claim 23 wherein the further immunomodulatory polypeptide acts as an adjuvant.

25. A method according to claim 23 wherein the further immunomodulatory polypeptide is an immunogenic pathogen derived sequence.

26. A method according to claim 25 wherein the immunogenic pathogen derived sequence is selected from the group consisting of tetanus toxoid fragment C (Frc) or component thereof, plant viral coat protein and cytokines.

27. A method according to claim 17 wherein antigen is derived from a pathogen.

28. A method according to claim 27 wherein the pathogen is selected from the group consisting of a virus and a bacteria.

29. A method according to claim 17 wherein the antigen is derived from a self or altered self-polypeptide.

30. A method according to claim 29 wherein the self or altered self-polypeptide is associated with an autoimmune disease.

31. A method according to claim 29 wherein the self or altered self-polypeptide is a tumor antigen.

32. A method according to claim 49 wherein the antigen selected from the group consisting of an antigen derived from colon carcimonaan antigen derived from a B-cell lymphoma, and an antigen derived from leukaemia.

33. A method of boosting an immune response in an individual to a tumor antigen, said individual having been previously primed against or exposed to said tumor antigen, said method comprising administering to said individual a nucleic acid construct encoding said antigen by electroporation.

34. A method according to claim 33 wherein the individual has been previously primed against said tumor antigen as a result of administration of said tumor antigen.

35. A method according to claim 34 wherein the antigen was previously administered to said individual as a nucleic acid construct encoding said antigen.

36. A method according to claim 34 wherein said tumor antigen was previously administered to said individual as a polypeptide comprising said tumor antigen.

37. A method according to claim 34, wherein the method comprises, prior to administering to said individual a nucleic acid construct encoding said antigen by electroporation, administering a priming tumor antigen to said individual.

38. A method according to claim 33 wherein the nucleic acid construct is selected from the group consisting of DNA, RNA and cDNA.

39. A method according to claim 33 wherein the nucleic acid construct encodes said tumor antigen and a further immunomodulatory polypeptide.

40. A method according to claim 39 wherein the further immunomodulatory polypeptide acts as an adjuvant.

41. A method according to claim 39 wherein the further immunomodulatory polypeptide is an immunogenic pathogen derived sequence.

42. A method according to claim 41 wherein the immunogenic pathogen derived sequence is selected from the group consisting of tetanus toxoid fragment C (Frc) or a component thereof, plant viral coat protein and cytokines.

43. A method according to claim 33 wherein the tumor antigen is selected from the group consisting of a tumor associated antigen and a tumor specific antigen.

44. A method according to claim 33 wherein the tumor antigen is selected from the group consisting of an antigen derived from colon carcinoma, an antigen is derived from a B-cell lymphoma, and an antigen derived from leukaemia.

45. A method according to claim 33 wherein the antigen is capable of inducing an immune response selected from the group consisting of a CD4+ T-cell response, a CD8+ T-cell response, a cytotoxic T lymphocyte (CTL) response and an antibody response.

46. A method of claim 33, wherein the induced immune response against a tumour antigen prevents or treats cancer in the individual.
